(19) United States
(12) Patent Application Publication
(10) Pub. No.: US 2020/0168298 A1
(43) Pub. Date: May 28, 2020

(54) GENOTYPING USING HIGH THROUGHPUT SEQUENCING DATA

(71) Applicant: Indiana University Research and Technology Corporation, Indianapolis, MA (US)

(72) Inventors: Suleyman Cenk Sahinalp, Bloomington, IN (US); Ibrahim Numanagic, Cambridge, MA (US); Salem Malikic, Burnaby (CA)

(21) Appl. No.: 16/615,089

(22) PCT Filed: May 21, 2018
(86) PCT No.: PCT/US2018/033740

§ 371 Date: Nov. 19, 2019

Related U.S. Application Data
(60) Provisional application No. 62/508,870, filed on May 19, 2017.

(51) Int. Cl. G16B 30/10; G16B 20/20; G06F 16/245

(52) U.S. Cl. CPC G16B 30/10; G06F 16/245; G16B 20/20

(57) ABSTRACT

Described herein are methods and systems for predicting the genotype of one or more genes utilizing high throughput sequencing data. The provided methods and systems allow for accurate genotyping of genes, including ADME genes, and can be used to identify novel alleles.

### CROSS-REFERENCE TO RELATED APPLICATIONS

This PCT application claims priority to U.S. Provisional Application No. 62/508,870, filed on May 19, 2017, the entire disclosure of which is expressly incorporated herein by reference for all purposes.

### STATEMENT OF GOVERNMENT SUPPORT

This invention was made with government support under GM108348 awarded by National Institutes of Health. The government has certain rights in the invention.

### FIELD

The present disclosure relates generally to methods and systems for predicting the genotype of one or more genes utilizing high throughput sequencing data.

### BACKGROUND

The use of genetic testing in personalized medicine is increasingly allowing for movement away from a standard of care that has been generalized for the general population and toward a more personalized, genome-based approached aimed at preventing, diagnosing, and treating disease in the individual.

The rapid development of high throughput sequencing (HTS) technologies has made a considerable impact on clinical genomics research. In principle, modern HTS platforms offer time-efficient, cost-effective and highly accurate means for genotyping clinically relevant genes. However, analyzing the sequencing data has posed problems in certain instances. Since many functionally and clinically important genes are highly polymorphic and have multiple copies as we as sequencewise-similar pseudogenes with which the frequently hybridize/fuse with to produce novel alleles, analyzing the sequence data is highly challenging from a computational point of view. In addition, some of these genes have been subject to structural alterations, making their allelic decomposition (i.e., determining the number of copies of a gene and the exact sequence content of each of its copies) computationally difficult.

Current computational tools are unable to utilize HTS data to perform allelic decomposition of genes that have been subject to structural alterations. Available structural variation detection tools aim to identify the type and locus of large “structure altering events” (e.g., large-scale deletions, novel sequence insertions, segmental duplications, and inversions), typically in uniquely mappable regions of the genome. In contrast, available copy number alteration detection/copy number phasing tools aim to identify the number of copies of a particular gene in each chromosome under the implicit assumption that the gene duplications or deletions always affect the entire (rather than a part of the) gene of interest, but do not reconstruct the exact sequence content of the gene. While certain methods have been developed that utilize small variants for copy number phasing, these methods are limited to detecting copy number changes only. They cannot also determine the exact sequence content of each copy of a gene that has been subject to structural alterations. No existing tool aims to find out what happens when structural alterations affect genes with multiple copies or those with highly homologous pseudogenes. Such genes are algorithmically difficult to resolve, as read that originate from such genes have high mapping ambiguity.

Those genes involved in the Absorption, Distribution, Metabolism, and/or Excretion (ADME) of pharmaceutical compounds are examples of highly polymorphic genes having multiple copies.

Accurately determining the genotypes of genes involved in the Absorption, Distribution, Metabolism, and/or Excretion (ADME) is essential to drug treatment and dosage decisions, and is highly recommended prior to treatment with certain drugs. Unfortunately, existing array-based genotyping assays are limited in scope in that they do not cover all genes and all potential variants of each gene, can be costly, and sometimes inaccurate.

Genotyping ADME genes can play an important role in identifying responders and non-responders to pharmaceutical compounds, avoiding adverse events, and optimizing drug dose, and assist treatment and dosage decisions for more than 90% of all prescribed drugs.

Targeted genotyping platforms, like Affymetrix DMET™ Plus arrays and the Illumina ADME assays are able to detect the common set of predefined variations and genotypes. However, rare variations are common across sites that impact drug response. The PGRNseq capture protocol was recently introduced, and offers a targeted sequencing platform for ADME gene. The PGRNseq protocol currently targets 84 ADME genes. Algorithmic challenges in exact ADME genotyping exist however, due to the presence of pseudo-genes, structural rearrangements, and copy number variation. This has resulted in a major roadblock to the use of HTS platforms in pharmacogenomics analysis. Additional obstacles such as the short read lengths offered by prominent sequencing technologies and non-uniformity of sequencing coverage for alternate sequencing technologies further complicate ADME genotyping.

### SUMMARY

The methods and systems described herein can be used to predict the genotype of one or more genes utilizing high throughput sequencing data. The provided methods and systems allow for accurate genotyping of genes, including ADME genes, and can be used to identify novel alleles.

In some embodiments, methods for genotyping a gene comprise receiving high throughput sequencing data for the gene from a target sample, wherein the high throughput sequencing data comprises a plurality of target sample reads; aligning the target sample reads of the high throughput sequencing data to one or more star-alleles of a reference genome allele database, wherein the reference genome allele database comprises nucleic acid sequences for known star alleles of the gene; identifying one or more nucleic acid sequence variants, or a lack of nucleic acid variants, in an allele of the gene relative to the one or more star-alleles of the reference genome allele database; detecting structural variants or a lack of structural variants in the allele; identifying one or more gene-disrupting mutations (i.e., functional mutations) or a lack of gene-disrupting mutations in the allele; selecting a set of one or more reference star alleles from the reference genome allele database that most closely match the identified one or more gene-disrupting mutations or a lack of gene-disrupting mutations in the allele; and calling, for an allele where a single star-allele was selected, a genotype associated with the selected star-allele.

In some embodiments, the method further comprises refining a genotype for an allele where two or more star-alleles were selected by assigning each of the selected star-alleles a penalty score based on minimizing a number of missing and additional non-functional variation in the allele in order to match the database as closely as possible, and calling a genotype for the allele as that genotype associated with one or more star-alleles having the lowest penalty score.

In some embodiments, the gene to be genotyped is an absorption, distribution, metabolism, and excretion (ADME) gene.

In some embodiments, the method is repeated for one or more additional genes.

In some embodiments, the high throughput data is targeted hybrid capture with consistent read distribution data or whole genome sequencing (WGS) data. In some embodiments, the hybrid capture with consistent read distribution data is PGRNseq data.

In some embodiments, the high throughput data is received as a FASTQ file, a uSAM file, or a uBAM file.

In some embodiments, alignment of target sample reads of the high throughput sequencing data is accomplished by BWA-MEM, BWA-backtrack, BWA-SW, LAST, Partek Flow, Bowtie 2, Stampy, SHRiMP2, SNP-o-matic, CLC Workbench, NextGenMap, Mosaik, ERNE-MAP, mrFAST, or mrsFAST-Ultra.

In some embodiments, alignment of target sample reads of the high throughput sequencing data comprises performing a local indel realignment.

In some embodiments, nucleic acid sequence variants are identified by FreeBayes, MuTect2, or SAMtools.

In some embodiments, the high throughput sequencing data coverage is consistent across samples but non-uniform across regions of the gene and sequencing depth is not known in advance.

In some embodiments, detecting structural variations in the allele comprises the steps of: estimating a gene copy number for one or more regions of the allele; determining an observed coverage for each of the one or more regions; and identifying an optimal gene arrangement by determining a minimal difference between the observed coverage for each of the one or more regions and coverage formed by one or more known possible gene arrangements, wherein a structural rearrangement is detected when the optimal gene arrangement is not a reference gene arrangement.

In some embodiments, identifying gene-disrupting mutations comprises the steps of comparing nucleic acid sequence variants identified in the allele and/or structural variation detected in the allele to the known alleles of the reference genome database.

In some embodiments, selecting a set of star-alleles that most closely match the identified one or more gene-disrupting mutations or lack of gene-disrupting mutations in the allele comprises the steps of: receiving a nucleic acid sequence for each known gene allele of the reference genome database; excluding nucleic acid sequences of known gene alleles that are not in agreement with a determined allele structural arrangement; excluding nucleic acid sequences of known gene alleles of the reference genome database that include neutral mutations; and selecting one or more known gene alleles of the reference genome database that most closely match the identified one or more gene-disrupting mutations or lack of gene-disrupting mutations in the allele.

In some embodiments, the methods described herein are executable using a suitably programmed computer. In some embodiments, the methods described herein improve the computational capacity of the suitably programmed computer.

Also described herein are systems for predicting a genotype of one or more genes. In some embodiments, a system comprises: a sample generator; a sequencer; at least one database having information regarding the one or more genes; and a sequence analyzer comprising a user interface and a system controller comprising at least one processer configured to perform the method according to an embodiment described herein. In some embodiments the at least one processor comprises a sequence aligner, a sequence variant identifier, a structural variant identifier, a gene-disrupting mutation identifier, a star-allele identifier, and a genotype caller.

In some embodiments, the sequence aligner is configured to align target sample reads of the high throughput sequencing data to a reference genome database; the sequence variant identifier is configured to identify nucleic acid sequence variants in a gene allele relative to the reference genome database; the structural variant identifier is configured to detect structural variants or a lack of structural variants in the gene allele; the gene-disrupting mutation identifier is configured to identify one or more gene-disrupting mutations or a lack of gene-disrupting mutations in the gene allele; the star-allele identifier is configured to identify one or more star-allele comprising the identified one or more gene-disrupting mutations or lack of gene-disrupting mutations; and the genotype caller is configured to determine the allele to have the genotype associated with the identified one or more star-alleles.

In some embodiments, elements of a system described herein are integrated into a standalone system located at a single site. In other embodiments, elements of a system described herein are located remotely with respect to each other.

### BRIEF DESCRIPTION OF THE DRAWINGS

FIG. 1 is a flowchart illustrating a method according to one embodiment.

FIG. 2A is a flowchart illustrating a method according to one embodiment.

FIG. 2B is a flowchart illustrating a method according to one embodiment.

FIG. 3 is a flowchart illustrating a method according to one embodiment.

FIG. 4A illustrates a representative example of PGRNseq coverage resealing for CYP2D6 according to one embodiment.

FIG. 4B illustrates a representative example of PGRNseq coverage resealing for CYP2D7 according to one embodiment.

FIG. 4C illustrates a representative example of PGRNseq coverage resealing for CYP2D8 according to one embodiment.

FIGS. 5A-5C illustrate the methods used for PGRNseq coverage normalization according to one embodiment, including a regular case (FIG. 4A), a deletion (FIG. 4B), and a fusion and duplication (FIG. 4C).

FIG. 6 depicts a family tree with the ADME genotype predictions for CYP2D6 for the CEPH 1463 family.

FIG. 7 is a block diagram illustrating a system formed in accordance with one embodiment that may be used to carry out the methods described herein.

Corresponding reference characters indicate corresponding parts throughout the several views

### DETAILED DESCRIPTION

The embodiments disclosed herein are not intended to be exhaustive or limit the disclosure to the precise forms disclosed in the following detailed description. Rather, the embodiments are chosen and described so that others skilled in the art may utilize their teachings.

Described herein is a tool and methods that can utilize HTS data to perform allelic decomposition of genes that have been subject to structural alterations. In some embodiments, the methods comprise i) finding out how many copies of a gene there are and which HTS read belongs to which copy (i.e., mapping ambiguity resolution), and ii) implicitly or explicitly assembling each copy of the gene from the read set (this is inherently intermingled with mapping ambiguity resolution) and identify the gene copy's origins relative to a reference genome. In some embodiments, the methods further comprise a) identifying all structural alteration breakpoints and carefully reconstructing the sequence content of each breakpoint region, while taking into account all micro-structural alterations, indels, and single nucleotide variants (SNVs) each copy of the gene has been subject to, and b) identifying fusions/hybridizations between the gene and its highly homologous pseudogenes. The tools and methods available to date fail to address these issues.

Existing structural variation discovery tools are based on the following general strategies: detection of variants using discordantly mapping paired-end reads (e.g., Variation Hunter (Hormozdiari, F. et al. (2009), Genome Res, 19:1270-1278; Hormozdiari, F. et al. (2010), Bioinformatics, 26:i350-i357) and Hydra (Quinlan, A R et al. (2010), Genome Res, 20:623-635), which report only the rough loci of structural variants but not their sequence content); detection of variants using split-read mappings (e.g., Socrates (Schroeder, J. et al. (2014), Bioinformatics, 40:1064-1072)); and detection of variants by mapping de novo assembled contigs to a reference genome (e.g., Barnacle (Swanson, L. et al. (2013), BMC Genomics, 14:550) and Dissect (Yorukoglu, D. et al. (2012), Bioinformatics, 28:i179-i187), which are RNA-Seq analysis tools that can also be used to analyze genomic data).

There are several tools available that employ a combination of these strategies (e.g., Pindel (Ye, K. et al. (2009), Bioinformatics, 25:2865-2871), Delly (Rausch, T. et al. (2012), Bioinformatics, 28:i333-i339), novoBreak (Chong, Z. et al. (2017), Nat. Methods, 14:65-67), and GASVPro (Sindi, S. et al. (2012), Genome Biol, 13:R22), which only consider uniquely mapped reads and cannot identify alterations in repetitive DNA. No available tool, even those designed to identify gene fusions only (e.g., defuse (McPherson, A. et al. (2011), PLoS Comput Biol, 7:1-16)), aims to reconstruct the sequence content of a fusion between a gene and a highly similar pseudogene. Further, no existing tool aims to infer variants from targeted capture sequencing data which are highly non-uniform in coverage (e.g., PGRNseq, which is discussed herein). Even tools that aim to genotype a particular gene such as the ADME gene CYP2D6, namely Cypiripi (Numanagic, I. et al. (2015), Bioinformatics, 31:i27-i34) and Astrolabe (formerly Constellation; Twist, G. P. et al. (2016), NPJ Genomic Med, 1:15007), respectively work only on uniform coverage sequencing data, or can determine the gene's sequence content only if it differs from a reference sequence by SNVs but not structural variation.

The methods provided herein address these challenges, and provide for the first time a framework to perform allelic decomposition of any gene of interest in HTS data. In some embodiments, the methods provided herein can perform allelic decomposition of any gene that differs from a reference genome by i) SNVs, ii) short indels, iii) full gene duplications or deletions (leading to copy number alteration), iv) partial gene duplications or deletions, as well as v) “balanced” fusions (i.e., those that preserve the structure of a gene) with highly homologous pseudogenes (the fusions can have one or more breakpoints). In some embodiments, all possible combinations of genomic alterations are identified, and the sequence content of all copies of a gene are determined in whole genome or targeted genome sequencing data.

Over 300 genes have been identified to participate in some way in the Absorption, Distribution, Metabolism and/or Excretion (ADME) of pharmaceutical compounds. Of these, 32 have been identified as core ADME genes, essential to ADME of pharmaceutical compounds. An additional 184 genes have been identified as related ADME genes, which includes those genes determined to be related to ADME of pharmaceutical compounds. ADME genotyping plays an important role in identifying responders and non-responders to pharmaceutical compounds, avoiding adverse events, and optimizing drug dose. Over 230 FDA-approved drugs provide pharmacogenomic information in their labeling.

Advances in DNA sequencing over the past two decades made it possible to explore the human genome in unprecedented detail. Whole genome sequencing (WGS) is now routinely performed in less than a day, and the Illumina HiSeq X sequencing system has driven the cost of WGS under $1,000 dollars per sample. Furthermore, Illumina-style WGS data offers high coverage depth, uniform read distribution and low error rates, all of which are useful for genotyping purposes. However, WGS is still considered costly and time-consuming compared to the targeted genotyping panels. Whole exome sequencing (WES) provides cheaper alternative to WGS, but in its current iteration, it is not able to sequence non-coding regions. This makes WES unsuitable for genotyping of ADME pharmacogenes, where variations in the non-coding regions can significantly affect phenotype.

Much of clinical genotyping is still performed through targeted genotyping panels. These targeted genotyping panels, like Affymetrix DMET™ Plus arrays and the Illumina ADME assays are able to detect a common set of predefined variations and genotypes. However, rare or personal variants, while functionally significant, often cannot be captured by these panels. Rare variants of pharmacogenes (e.g., CYP2D6) can impact drug response. As a result, new HTS-based targeted captures are being introduced to help identify novel variants in a cost-effective manner. A prime example is the PGRNseq capture protocol, which was recently introduced and offers a targeted sequencing platform for ADME gene. The PGRNseq protocol currently targets 84 ADME genes (Table 1; see Gordon, A. S., et al., (April 2016) Pharmacogenet Genomics 26(4):161-168 (Epub January 2016)), which is hereby incorporated by reference in its entirety). For each of these genes, PGRNseq covers at least its exonic region and a few kilobases upstream and downstream of gene's untranslated region (UTR), covering more than 960 KB of the human genome through its first iteration (PGRNSeq v.1). PGRNseq maintains backward-compatibility with previous DMET™ Plus and Illumina ADME assays by targeting all single nucleotide variations (SNV) included in those panels. In total, more than 960 KB of genome is covered by PGRNseq. PGRNseq capture products are sequenced on the Illumina HiSeq 2000 platform, which provides low error rates while maintaining very high depth of coverage (averaging 500× per chromosome). Most importantly, PGRNseq is significantly less expensive than WGS and even WES. For example, PGRNseq is up to ten times less expensive than WGS. Thus, PGRNseq offers a competitively-priced platform for clinical genotyping of targeted genes, while providing all the benefits of standard WGS sequencing. And even though PGRNSeq (or WGS) data from some of the pharmacogenes are relatively straightforward to interpret, other, more difficult genes such as CYP2D6 have proven difficult to analyze.

PGRNseq inherits some of the problems that come with WGS, including short read length and data interpretation issues. Genotype inference for ADME genes harboring various structural rearrangements still presents a major challenge. In order to assist the analysis of such structural variants, the second iteration of PGRNseq covers the whole genic clusters containing the targeted ADME genes (e.g., for CYP2D6, the entire 30 KB CYP2D cluster is sequenced, which includes CYP2D6 and pseudogenes CYP2D7 and CYP2D8).

The present disclosure provides the first computational tool to exactly genotype ADME genes based on PGRNseq and Illumina WGS data, with the capability to accurately handle gene duplications, fusions, and genomic deletions. Available CYP2D6 genotyping tools such as Cypiripi (see Numanagic, I. et al. (2015), Bioinformatics, 31:i27-i34 and Astrolabe (see Twist, G. P. et al. (2016), NPJ Genomic Med, 1:15007) are not only limited to uniform coverage WGS data, but are also unable to properly detect some structural rearrangements. And while genotyping CYP2D6 is valuable in that its encoded enzyme is involved in metabolism of 20-25% of clinically prescribed drugs, neither of these tools provide support for genotyping other ADME genes. Previous attempts to analyze PGRNseq data, which relied on SNP callers to infer genotype, resulting in Mendelian inconsistencies (see Gordon, A. S. et al. (April 2016), Pharmacogenet Genomics, 26(4):161-168 (Epub January 2016)). The methods and systems described herein are demonstrated on a large selection of WGS and PGRNseq samples to be a highly accurate and very fast tool for ADME genotyping.

The tool and methods described herein is not limited to the genotyping of ADME genes, and can be applied broadly to any gene or group of genes. While embodiments and examples provided herein describe the genotyping of ADME genes, the described methods can be similarly applied to other genes by those of skill in the art having the benefit of the present disclosure.

The tool and the methods described herein are capable of reconstructing the structure and sequence content of each copy of a particular gene present in a sample being analyzed. Following the well-established star-allele nomenclature in pharmacogenomics, a star-allele of a gene is defined as a gene sequence which differs from the “wild type” (or canonical) gene sequence by a (non-empty) set of mutations. Thus, reconstructing the sequence content of a gene copy is identical to identification of the gene copy's star-allele, which could either be already known or possibly novel.

Two types of mutations, and as a consequence, star-alleles, are distinguished. Any mutation that has an impact on the resulting protein product of the gene is referred to herein as a “gene-disrupting mutation” (also known as a “functional mutation”). Gene-disrupting mutations include codon-changing single nucleotide polymorphisms (SNPs) and indels, as well as mutations outside the coding regions that affect the protein enzyme activity. Star-alleles which are defined solely by gene-disrupting mutations are referred to as “major star-alleles,” and are assigned a unique number. For example the canonical “wild type” star-allele is always assigned *1, while *2 describes a star-allele that harbors one or more gene-disrupting mutations capered to the *1. If a new major star-allele is discovered that has not previously been reported in the literature, new star allele is identified by *nl+1, where l is the number of major star-alleles known up to that point. It is possible that two major star-alleles can share a common mutation.

A mutation that does not impact the protein product is referred to herein as a “neutral mutation” (also known as a “non-functional mutation”). Any major star-allele can be extended with neutral mutations, and such extension is referred to as a “minor star-allele.” If a copy of a gene includes only neutral mutations, then it is considered to be an extension of the wild-type star-allele. In order to distinguish various minor star-alleles, a unique symbol, and in certain instances, a pair of symbols, is attached to the major star-allele's number for each such extension. For example, minor star-allele 2A is formed by taking the set of gene-disrupting mutations for major *2 allele and extending it with some neutral mutation; *2B is formed in a similar manner, however the sets describing the neutral mutations of *2A and *2B are not identical, although the sets describing their gene-disrupting mutations are. If a new minor star-allele that is an extension to the star-allele *κ is discovered, it is commonly called *κX where X is the lexicographically smallest letter which has not yet been used for minor alleles of *κ.

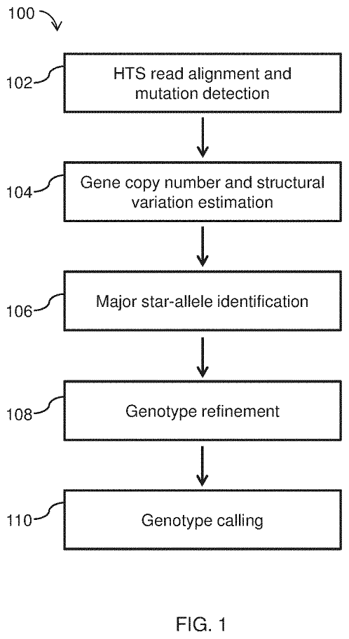

In view of these definitions, the tool and methods described characterize the sequence composition of each copy of a gene present in a sample, which is by definition equivalent to inferring the major and minor star-allele label of such gene copy. Where there is a need to define a new star-allele, this is done by minimizing the number of novel mutations and structural variations that need to be added to or subtracted from a known star-allele to describe the new star-allele. In certain aspects, and as depicted in FIG. 1, the methods for genotyping 100 (i.e., characterizing the sequence composition of a gene) comprise the steps of: 1) read alignment and mutation detection 102, where high throughput sequencing (HTS) reads are aligned to a reference genome and mutations present in a target gene region are identified; 2) copy number and structural variation estimation 104, where copy number of the gene is identified, and if present, various structural variations are identified; 3) major star-allele identification 106, where the major star-allele of each gene copy is established; 4) genotype refinement 108, where the supporting set of neutral mutations is assigned to each major star-allele, and the “score” of such an assignment (see “Genotype Refining” section) is used to rank each allelic configuration identified; and 5) genotype calling 110, where the final genotype (i.e. minor star-allele) is obtained by choosing the set of allelic configurations with the best ranking score. In instances where multiple configurations have the same score, all configurations will be reported as equally likely genotypes.

In some embodiments, the primary input is HTS in SAM/BAM file format, as well as one or more databases comprised of information about the gene to be genotyped. Such databases will contain basic information about the gene (e.g., its location within a reference genome, locations of pseudogenes, intron/exon boundaries), as well as a listing of all known major and minor star-alleles for that gene. Each described as a unique set of gene-disrupting and neutral mutations. The databases will also contain a listing of all known structural variations involving the gene of interest; i.e., duplications and deletions, as well as all known hybridizations with its pseudogene, either in the form of fusions (when a prefix or suffix of the hybrid gene sequence is from the pseudogene) or gene conversions (when a segment other than a suffix/prefix of the hybrid gene is from the pseudogene). In some embodiments, all information about the gene to be genotyped is maintained by and obtained from a single database. In some embodiments, the information about the gene to be genotyped is maintained by and obtained from two or more separate databases.

The tool and methods described herein utilize the gene information from the one or more databases to “guide” star-allele discovery, aiming to assign a known major and minor star-allele label for each copy of the gene. In instances where no known star-allele description “matches” the input data, the tool and methods described will infer previously unknown major or minor star-allele descriptions. Mutations considered herein include SNVs and short indels; the structural variation considered include (partial) deletions or duplications of the gene, and hybridizations (a.k.a fusions) with a specified pseudogene.

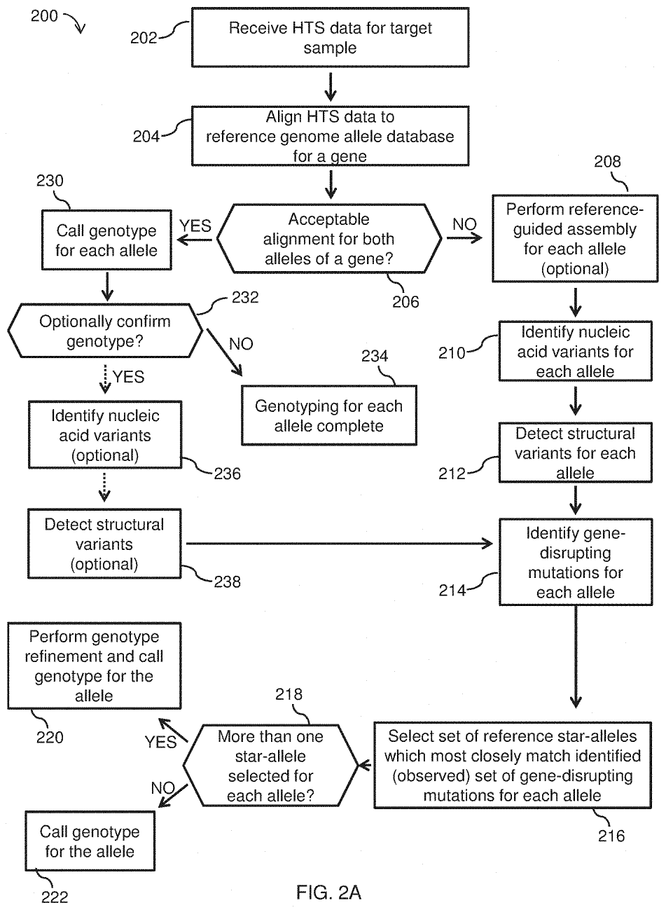

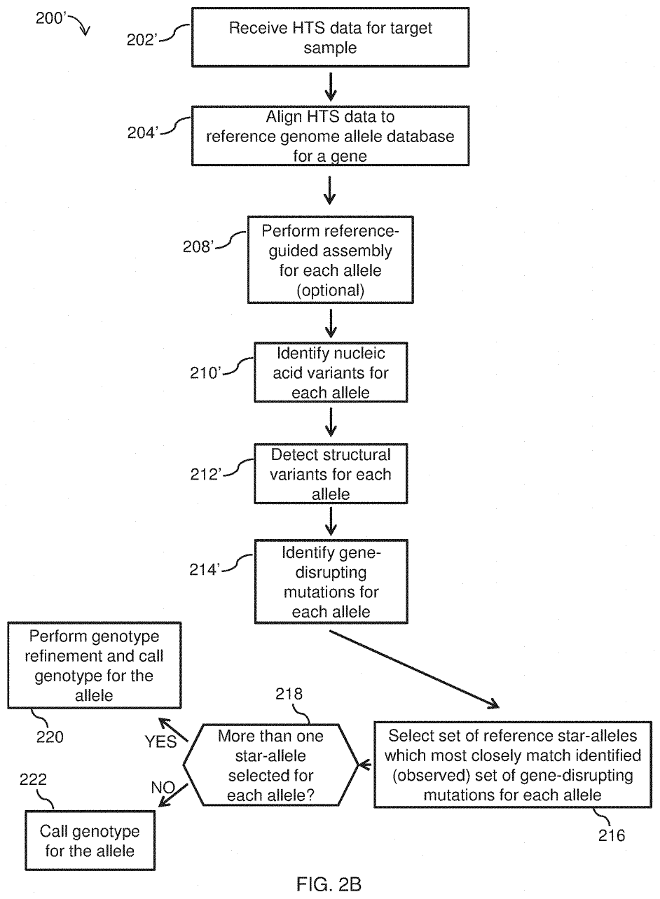

FIG. 2A depicts one embodiment of methods for genotyping 200, which follows the general method 100 of FIG. 1. As depicted in FIG. 2A, in some embodiments, methods for genotyping 200 comprise the steps of: 1) receiving high throughput sequencing (HTS) data for a gene from a target sample 202, 2) aligning target sample reads from the HTS data to a reference genome allele database for the gene 204, 3) determining whether the alignment is acceptable for both alleles of the gene 206, and if yes, 4) calling the genotype for each allele 230, but if no, 5) identifying nucleic acid variants for each allele 210, 6) detecting structural variants or a lack of structural variants in each allele 212, 7) identifying one or more gene-disrupting mutations or a lack of gene-disrupting mutation in each allele 214, 8) for each allele, selecting a set of reference star-alleles which most closely match the identified (i.e., observed) set of gene-disrupting mutations in each allele, and 9), determining each allele of the gene to have the genotype associated with the identified set of reference star-alleles for that allele. In some embodiments, the set of reference star-alleles can include one or more different star-alleles. In some embodiments, the set of reference star-alleles includes a single star allele. In these embodiments, an allele genotype can be called based on that single star-allele 222. In other embodiments, the set of reference star-alleles includes two or more star alleles. In these embodiments, the genotyping method 200 can further comprise a genotype refining step 220. In some embodiments, the genotype refining step 220 comprises ranking each possible solution, or identified star-allele in the set of reference star-alleles, and the one or more solutions with the best ranking score are identified as the genotype for the allele. In some embodiments, the method for genotyping is repeated for each gene of interest. In some embodiments, one or more steps of the methods for genotyping are performed by a suitably programmed computer. In some embodiments, one or more genes of interest are genotyped simultaneously. Each of the steps is described herein in detail.

As depicted in FIG. 2A, in some embodiments, a method for genotyping a gene comprises receiving HTS data for a gene from a target sample 202, aligning target sample reads from the HTS data to gene information from one or more reference databases for the gene 204, determining whether the alignment is acceptable for both alleles of the gene 206, and where the alignment is determined to be acceptable for each allele, calling the genotype for each allele 230. The genotype of each allele can optionally be confirmed 232. In some embodiments, the genotype of each allele is not confirmed, and the genotyping for each allele of the gene is complete 234. In other embodiments, the genotype of each allele is optionally confirmed. Where the genotype of each allele is to be confirmed, an allele sequence may optionally be surveyed to identify nucleic acid variants 236 and detect structural variants 238. If the optional steps of identifying nucleic acid variants 236 and detecting structural variants 238 are carried out, gene-disrupting mutations for each allele are identified 214. In some embodiments, gene-disrupting mutations for each allele are identified 214 without identifying nucleic acid variants 236 or detecting structural variants 238 in the sequence of each allele. Where an acceptable alignment for both alleles has been achieved, these two optional steps can be skipped, as the exact sequence for each allele, and thus any nucleic acid variant or structural variant, is known. Following identification of gene-disrupting mutations for each allele 214, a set of reference star-alleles that most closely match the identified (i.e., observed) set of gene-disrupting mutations for each allele is selected 216, and it is determined whether more than one reference star-allele was selected for each allele 218. In some embodiments, the set of reference star-alleles can include one or more star-alleles. In some embodiments, the set of reference star-alleles includes a single star-allele. In these embodiments, an allele genotype can be called based on that single reference star-allele 222. In other embodiments, the set of reference star-alleles includes two or more star-alleles. In these embodiments, the genotyping method can further comprise a genotype refining step 220. In some embodiments, the genotype refining step 220 comprises ranking each possible solution, or identified allele in the set of reference star-alleles, and the one or more solutions with the best ranking score are identified as the genotype for the allele.

As depicted in FIG. 2A, in some embodiments, an acceptable alignment for both alleles of a gene is not present at step 206. In such embodiments, further reference-guided assembly of the HTS data for each allele is performed 208. Following alignment of the HTS data for each allele, nucleic acid variants are identified 210, structural variants are detected 212, and gene-disrupting mutations for each allele are identified 214. Following identification of gene-disrupting mutations for each allele 214, a set of reference star-alleles that most closely match the identified (i.e., observed) set of gene-disrupting mutations for each allele is selected 216, and it is determined whether more than one reference star-allele was selected for each allele 218. In some embodiments, the set of reference star-alleles can include one or more star-alleles. In some embodiments, the set of reference star-alleles includes a single star-allele. In these embodiments, an allele genotype can be called based on that single reference star allele 222. In other embodiments, the set of reference star-alleles includes two or more star-alleles. In these embodiments, the genotyping method can further comprise a genotype refining step 220. In some embodiments, the genotype refining step 220 comprises ranking each possible solution, or identified star-allele in the set of reference star-alleles, and the one or more solutions with the best ranking score are identified as the genotype for the allele.

As depicted in FIG. 2B, in some embodiments a genotyping method 200′ ignores whether an acceptable alignment for both alleles of a gene is found. In such embodiments, reference-guided assembly of the HTS data for each allele is performed 208′. Following alignment of the HTS data for each allele, nucleic acid variants are identified 210′, structural variants are detected 212′, and gene-disrupting mutations for each allele are identified 214′. Following identification of gene-disrupting mutations for each allele 214′, a set of reference star-alleles that most closely match the identified (i.e., observed) set of gene-disrupting mutations for each allele is selected 216′, and it is determined whether more than one reference star-allele was selected for each allele 218′. In some embodiments, the set of reference star-alleles can include one or more star-alleles. In some embodiments, the set of reference star-alleles includes a single star-allele. In these embodiments, an allele genotype can be called based on that single star-allele 222′. In other embodiments, the set of reference star-alleles includes two or more star-alleles. In these embodiments, the genotyping method can further comprise a genotype refining step 220′. In some embodiments, the genotype refining step 220′ comprises ranking each possible solution, or identified star-allele in the set of reference star-alleles, and the one or more solutions with the best ranking score are identified as the genotype for the allele.

### CYP2D6

CYP2D6 is one of the most widely studied pharmacogenes, and is involved in the metabolism of approximately 20-25% of all clinically prescribed drugs. CYP2D6 is located in the vicinity of highly homologous and evolutionarily related pseudogenes that facilitate formation of various structural rearrangements and copy number gains or losses, resulting in gene variants that affect a patient's drug metabolism. Genotype inference for ADME genes such as CYP2D6 and CYP2A6 still presents a major challenge.

Structural variations of CYP2D6, which include gene deletion, duplications and fusions with neighboring CYP2D7, can significantly affect CYP2D6 enzyme activity. Cytochrome P450 2A6 (CYP2A6) also metabolizes several clinically used drugs, but more importantly, it is the principle metabolizer of nicotine and its by-product cotinine. It has been suggested that CYP2A6's genotype is correlated with lower smoking risks, decreased cigarette consumption and increased cessation Similar to CYP2D6, CYP2A6 is highly polymorphic, with more than 50 alleles observed so far. It harbors various gene duplications and crossovers with highly homologous neighboring pseudogene CYP2A7. While genotyping of CYP2D6 can predict a patient's therapy response, genotyping CYP2A6 can shed light into a patient's smoking behavior.

Many of the models and equations of the various embodiments described herein utilize CYP2D6 as a representative ADME gene, which as described supra, are themselves examples of a group of gene that can be genotyped using the methods described herein. It will be recognized that the models and equations described herein can be applied to other ADME genes, as well as other groups of genes. In addition to CYP2D6, the methods of embodiments described herein have been applied to successfully genotype ADME genes CYP2A6, CYP2C19, CYP2C8, CYP2C9, CYP3A4, CYP3A5, CYP4F2, CYP4F2, TPMT, and DPYD (see Tables 4 and 5), each of which is a core ADME gene, with the exception of CYP4F2, which is on the list of related ADME genes.

In some embodiments, various modifications can be made to the models and equations without departing from the scope of the present disclosure. Applications of the models and equations to other genes, as well as any modification thereof, are deemed to be within the spirit, scope and concept of the present disclosure.

### HTS Data

In some embodiments, HTS sequencing data for an ADME gene is received in a suitable format such as a FASTQ file, a SAM file, or a BAM file. In some embodiments, the SAM or BAM file is an unaligned file (uSAM or uBAM). In some embodiments, the HTS sequencing data comprises data for one or more ADME genes. In some embodiments, the HTS sequencing data comprises data for the ADME genes included in the PGRNseq protocol (Table 1), although genes can be removed and/or other ADME genes added to the list of genes.

TABLE 1
ADME genes included in the PGRNseq protocol

| Gene Symbol | Gene Name |
|---|---|
| ABCA1 | ATP-binding cassette, sub-family A (ABC1), member 1 |
| ABCB1 | ATP-binding cassette, sub-family B (MDR/TAP), member 1 |
| ABCB11 | ATP-binding cassette, sub-family B (MDR/TAP), member 11 |
| ABCC2 | ATP-binding cassette, sub-family C (CFTR/MRP), member 2 |
| ABCG1 | ATP-binding cassette, sub-family G (WHITE), member 1 |
| ABCG2 | ATP-binding cassette, sub-family G (WHITE), member 2 |
| ACE | Angiotensin I converting enzyme |
| ADRB1 | Adrenoceptor beta 1 |
| ADRB2 | Adrenoceptor beta 2 |
| AHR | Aryl hydrocarbon receptor |
| ALOX5 | Arachidonate 5-lipoxygenase |
| APOA1 | Apolipoprotein A1 |
| ARID5B | AT-Rich interacting domain 5B |
| BDNF | Brain derived neurotrophic factor |
| CACNA1C | Calcium voltage-gated channel subunit alpha1 C |
| CACNA1S | Calcium voltage-gated channel subunit alpha1 S |
| CACNB2 | Calcium voltage-gated channel auxiliary subunit beta 2 |
| CES1 | Carboxylesterase 1 |
| CES2 | Carboxylesterase 2 |
| COMT | Catechol-O-methyltransferase |
| CRHR1 | Corticotropin releasing hormone receptor 1 |
| CYP1A2 | cytochrome P450, family 1, subfamily A, polypeptide 2 |
| CYP2A6 | cytochrome P450, family 2, subfamily A, polypeptide 6 |
| CYP2B6 | cytochrome P450, family 2, subfamily B, polypeptide 6 |
| CYP2C19 | cytochrome P450, family 2, subfamily C, polypeptide 19 |
| CYP2C9 | cytochrome P450, family 2, subfamily C, polypeptide 9 |
| CYP2D6 | cytochrome P450, family 2, subfamily D, polypeptide 6 |
| CYP2R1 | Cytochrome P450, family 2, subfamily R, polypeptide 1 |
| CYP3A4 | cytochrome P450, family 3, subfamily A, polypeptide 4 |
| CYP3A5 | cytochrome P450, family 3, subfamily A, polypeptide 5 |
| DBH | Dopamine beta-hydroxylase |
| DPYD | dihydropyrimidine dehydrogenase |
| DRD1 | Dopamine receptor D1 |
| DRD2 | Dopamine receptor D2 |
| EGFR | Epidermal growth factor receptor |
| ESR1 | Estrogen receptor 1 |
| FKBP5 | FK506 binding protein 5 |
| G6PD | Glucose-6-phosphate dyhydrogenase |
| GLCCI1 | Glucocorticoid induced 1 |
| GRK5 | G protein-coupled receptor kinase 5 |
| HMGCR | 3-hydroxy-3-methylglutaryl-CoA reductase |
| HSD11B2 | Hydroxysteroid 11-beta dehydrogenase 2 |
| HTR1A | HtrA serine peptidase 1 |
| HTR2A | HtrA serine peptidase 2 |
| KCNH2 | Potassium voltage-gated channel subfamily H member 2 |
| LDLR | Low density lipoprotein receptor |
| MAOA | Monoamine oxidase A |
| NAT2 | N-acetyltransferase 2 (arylamine N-acetyltransferase) |
| NPPB | Natriuretic peptide B |
| NPR1 | Natriuretic peptide receptor 1 |
| NR3C2 | Nuclear receptor subfamily 3, group C, member 2 |
| NTRK2 | Neurotrophic receptor tyrosine kinase 2 |
| PEAR1 | |
| POR | Cytochrome p450 oxidoreductase |
| PTGIS | Prostaglandin I2 synthase |
| PTGS1 | Prostaglandin-endoperoxide synthase 1 |
| RYR2 | Ryanodine receptor 2 |
| SCN5A | Sodium voltage-gated channel alpha subunit 5 |
| SLC15A2 | solute carrier family 15 (H+/peptide transporter), member 2 |
| SLC22A1 | solute carrier family 22 (organic cation transporter), member 1 |
| SLC22A2 | solute carrier family 22 (organic cation transporter), member 2 |
| SLC22A3 | Solute carrier family 22 (organic cation transporter), member 3 |
| SLC22A6 | solute carrier family 22 (organic anion transporter), member 6 |
| SLC47A1 | solute carrier family 47, member 1 |
| SLC47A2 | solute carrier family 47, member 2 |
| SLC6A3 | solute carrier family 6, member 3 |
| SLC6A4 | solute carrier family 6, member 4 |
| SLCO1A2 | solute carrier organic anion transporter family, member 1A2 |
| SLCO1B1 | solute carrier organic anion transporter family, member 1B1 |
| SLCO1B3 | solute carrier organic anion transporter family, member 1B3 |
| TBXAS1 | Thromboxane A synthase 1 |
| TCL1A | t-cell leukemia/lymphoma 1A |
| TPMT | thiopurine S-methyltransferase, |
| UGT1A1/4 | UDP glucuronosyltransferase 1 family, polypeptide A1 |
| VDR | Vitamin D receptor |
| VKORC1 | Vitamin K epoxide reductase complex subunit 1 |
| ZNF423 | Zinc finger protein 423 |

The HTS sequencing data can be generated for the selected ADME genes by methods known in the art. In some embodiments, one or more ADME genes are sequenced using the PGRNseq protocol (see Gordon, A. S., et al., (April 2016) Pharmacogenet Genomics 26(4):161-168 (Epub January 2016)). In other embodiments, WGS, which includes sequence data for all ADME genes, is performed. Any WGS sequencing system and protocol can be used. In some embodiments, the WGS sequencing system is the Illumina HiSeq X sequencing system. Any sequencing system used will generate at least one of a FASTQ file, a uSAM file, or a uBAM file. Where the sequencing system generates a FASTQ file, a uSAM or uBAM file can be generated therefrom. In some embodiments HTS sequencing data for one or more ADME genes is received in a uBAM file.

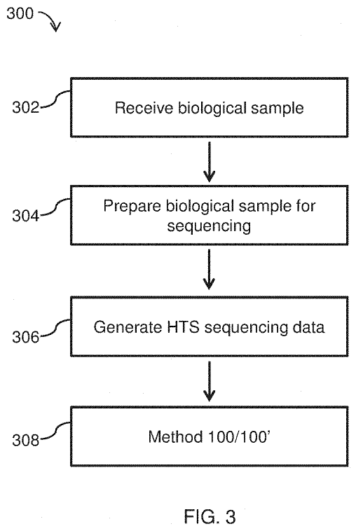

FIG. 3 illustrates a method 300 wherein a sample is sequenced prior to analysis. Such methods comprise obtaining or receiving a biological sample 302, preparing the biological sample for sequencing 304, generating HTS data from the biological sample 306, and performing one of methods 100, 200, or 200′, each of which are described herein.

In some embodiments, the HTS sequencing data is aligned to a reference genome allele database. In some embodiments, a reference genome allele database comprises nucleic acid sequences of all known alleles for one or more genes, such as, for example, ADME genes. In some embodiments, the reference genome allele database is obtained from an existing genome allele database. In other embodiments, the reference genome allele database is curated or compiled from two or more existing databases. For example, a reference genome allele database can be obtained or compiled from a variant database such as the Human Cytochrome P450 (CYP) Allele Nomenclature Database (available at cypalleles.ki.se), the Leiden Open Variation Database (available at databases.lovd.nl/shared/genes; includes allele variant data for many ADME genes, a gene-specific database such as The Human Cytochrome P450 Allele Nomenclature Database (available at cypalleles.ki.se/), or a general genomic database such as Gene, which is maintained by the National Center for Biotechnology Information (available at ncbi.nlm.nih.gov/gene).

### Alignment/Read Mapping

In some embodiments, alignment to each allele sequence of the reference genome allele database is carried out by a sequence alignment algorithm. In some embodiments, the HTS data is aligned to the sequence of each allele of the reference genome allele database simultaneously. In some embodiments, the HTS data for multiple genes is aligned to the sequence of each allele of a reference genome allele database for each gene, simultaneously.

In some embodiments, relevant reads are mapped to a reference genome by following a “best practices workflow” involving a read mapper (e.g., BWA (Li, H. & Durbin, R. (2009), Bioinformatics, 25:1754-1760) and CORA (Yorukoglu, D. et al. (2016), Nat Biotechol, 34:374-376)) and Genome Analysis Toolkit (GATK; McKenna A. et al. (2010), Genome Res, 20:1297-1303). The GATK pipeline performs local indel realignment, which can improve the detection of small indels. However, the tool and methods described herein are not limited to this mapping framework. Any valid SAM/BAM file containing the region of the targeted gene can be used.

Many algorithms are available capable of sequence alignment and include, for example, BWA-MEM (Li, H. (2013) arXiv: 1303.3997v1 [q-bio.GN]; available at bio-bwa.sourceforge.net/), BWA-backtrack available at bio-bwa.sourceforge.net/), BWA-SW (Li, H., and Durbin, R. (2010) Bioinformatics, 26:589-595; available at bio-bwa.sourceforge.net/), LAST (available at last.cbrc.jp/), Partek Flow (available at partek.com), Bowtie 2 (Langmead, B., and Salzberg, S. (2012) Nature Methods, 9:357-359; available at http://bowtie-bio.sourceforge.net/bowtie2/index.shtml), Stampy (Lunter and Goodson (2011) Genome Res, 21:936-939; available at well.ox.ac.uk/project-stampy), SHRiMP2 (David, M., et al. (2011) Bioinformafics, 27(7):1011-1012; available at compbio.cs.toronto.edu/shrimp/), SNP-o-matic (Manske, H. M., and Kwiatkowski, D. P. (2009) Bioinformafics, 25(18):2434-2435; available at github.com/magnusmanske/snpomatic), CLC Workbench (available at qiagenbioinformatics.com/products/clc-main-workbench/), NextGenMap (Sedlazeck, F. J., et al. (2013) Bioinformafics, 29(21):2790-2791; available at cibiv.github.io/NextGenMap/), Novoalign (available at novocraft.com/products/novoalign/), Mosaik (Lee, W. P., et al. (2014) PLOS One, 9(3):e90581; available at github.com/wanpinglee/MOSAIK), ERNE-MAP (available at erne.sourceforge.net/), mrFAST (Alkan, C., et al. (2009), Nature Genetics, October, 41(10):1061-1067, 2009), and mrsFAST-Ultra (Hach, F., et al. (2014) Nucleic Acids Res, 42(W1):W494-W500). Improvements or updates to these representative algorithms, or other sequence alignment algorithms that may exist or that may be developed, can also be used. The advantages of each of the representative alignment algorithms provided herein will be recognized, and an appropriate algorithm can be selected for use in a particular situation. For example, factors such as an algorithm's alignment accuracy, read length requirement, speed, computational requirements, and application-specific alignment features (e.g., paired-end alignment, gapped alignment, indel realignment, trimming alignment, and bisulfite alignment) can be considered when selecting an alignment algorithm.

In some embodiments, alignment of the HTS sequence data to the reference genome allele database(s) comprises reference-guided assembly. In some embodiments, reference-guided assembly is performed using a wild-type allele sequence.

In some embodiments, the alignment algorithm used performs a local indel (insertion-deletion) realignment. In some embodiments, the indel realignment improves detection of various small indels during the step of identifying nucleic acid variants.

In some embodiments, an acceptable alignment relative to star-alleles of the reference genome allele database for both alleles of a gene may be achieved. In such embodiments, the HTS data for each allele of a target gene lines up sufficiently with a star-allele of the reference genome allele database to allow for the genotype of both alleles to be called as the star-allele of the reference genome allele database to which the alleles of the target gene align. In some embodiments, an “acceptable alignment” refers to a nucleic acid sequence alignment between the nucleic acid sequence of an allele of a target gene and a nucleic acid sequence of an allele of the reference genome allele database having 98% to 100% sequence identity and at least 50% of expected overall coverage. In some embodiments, an “acceptable alignment” refers to two nucleic acid sequences having 100% sequence identity, with the HTS data having at least 50% of expected coverage.

### Sequence Variant Calling

In some embodiments, alignment of the HTS data allows for the identification of various nucleic acid variants. Such variants are identified, or called, by deviations in nucleic acid sequence relative to the reference genome sequence. In some embodiments, the variant is a single nucleotide variant (SNV), an insertion or deletion of one or more nucleic acids (indel), a multi-nucleotide polymorphism (MNP), or a composite insertion and substitution. Algorithms for calling nucleic acid variants are available and include, for example, FreeBayes (Garrison, E., and Marth, G. (2012) arXiv preprint arXiv:1207.3907 [q-bio.GN]; available at github.com/ekg/freebayes), MuTect2 (DePristo, M., et al. (2011) Nature Genetics, 43:491-498 and Van der Auwera, G. A., et al. (2013) Current Protocols in Bioinformatics, 43:11.10.1-11.10.33; available at software.broadinstitute.org/gatk/), and SAMtools (available at samtools.sourceforge.net/). Improvements or updates to these representative algorithms, or other nucleic acid variant calling algorithms that may be developed, can also be used.

In some embodiments, no nucleic acid variants are identified in the HTS data from the gene. In such embodiments, it is likely that the gene has the same genotype as the reference genome.

In some embodiments, the alignment step and the identification of nucleic acid variants step can be completed using the Genome Analysis Toolkit's (GATK) Best Practices workflow (see Li, H. (2013) arXiv:1303.3997v1 [q-bio.GN], DePristo, M., et al. (2011) Nature Genetics, 43:491-498; Van der Auwera, G. A., et al. (2013) Current Protocols in Bioinformatics, 43:11.10.1-11.10.33; available at software.broadinstitute.org/gatk/; and McKenna, A. et al. (2010), Genome Res, 20:1297-1303).

In some embodiments, identified nucleic acid variants can be received in a Variant Call Format (VCF) file. In such embodiments, nucleic acid variants have already been identified. In some embodiments, the variants identified in the VCF file are accepted as-is. In other embodiments, the variants identified in the VCF file are confirmed and either accepted or rejected.

### Detecting Structural Variants

ADME genotyping methods described herein comprise the step of detecting structural variants, or a lack thereof, in the ADME gene for which the HTS data is provided. In some embodiments, this step comprises estimating gene copy number for one or more regions of the gene, determining the observed coverage for each of the one or more regions of the gene, and identifying an optimal gene arrangement by determining a minimal difference between the observed coverage for each of the one or more regions and coverage formed by one or more known possible gene arrangements, where a structural rearrangement is detected when the optimal gene arrangement differs from the reference genome arrangement. In some embodiments, known possible gene arrangements are obtained from databases such as the Human Cytochrome P450 (CYP) Allele Nomenclature Database (available at cypalleles.ki.se), the Leiden Open Variation Database (available at databases.lovd.nl/shared/genes), which includes variant data for many ADME genes, a gene-specific database such as The Human Cytochrome P450 Allele Nomenclature Database (available at cypalleles.ki.se/), or a general genomic database such as Gene, which is maintained by the National Center for Biotechnology Information (available at ncbi.nlm.nih.gov/gene). In some embodiments, the database can be a unique database comprising curated data for one or more ADME genes.

Several ADME genes including CYP2D6 are prone to copy number variations, including gene deletions duplications and duplications, typically producing exactly two copies of a gene in a chromosome; cases in which more than two copies are present are sometimes called multiplications. Some genes, such as CYP2D6, are also prone to structural variations/alterations in the form of deletions and duplications that affect only part of the gene. For example, high sequence homology and close physical proximity between CYP2D6 and related pseudogene CYP2D7 results in the formation of gene conversions, producing hybrid genes where a prefix or suffix of the hybrid gene is from the pseudogene CYP2D7, or gene conversions, where one or more non-prefix/suffix contiguous segment of the hybrid gene is from CYP2D7. Some genes, including CYP2D6, have more than one pseudogene, however typically only one such pseudogene is highly homologous and difficult to distinguish. In many cases, the existence of structural variations has a substantial impact on the resulting protein products, making accurate characterization important for proper phenotype prediction. While the following description will use CYP2D6 as a representative gene, all models and calculations can be similarly applied to other ADME genes.

Gene duplications and deletions typically impact a gene such as CYP2D6 or a pseudogene such as CYP2D7 as a whole. In the case where such a deletion or a duplication impacts only part of a gene or a pseudogene, it is called a partial gene deletion or a partial gene duplication. As all structural variations/alterations resulting in the formation of hybrid genes are defined at the level of whole introns and exons, it is assumed that partial gene deletions or partial gene duplications involve one or more contiguous sequence(s) of whole introns and exons. As a result, it is assumed that each exon and each intron of the hybrid gene originates from either CYP2D6 or CYP2D7 as a whole.

“η” if is the total number of exons and introns in a gene, as well as in its highly related pseudogene (under the assumption that the number of exons and introns are conserved in the pseudogene). “R=[r₁, r₂, . . . , rₙ, rₙ₊₁, rₙ₊₂, r₂ₙ,]” denotes the sequence of introns and exons of the gene and pseudogene (i.e., CYP2D6 and CYP2D7), where r₁ stand for the first intron and r₂ stand for the first exon of CYP2D6 (n=19 for CYP2D6) Similarly, rₙ₊₁ and rₙ₊₂, respectively, stand for the first intron and the first exon of CYP2D7. Each hybrid configuration of CYP2D6/2D7 is represented by a vector v of length 2n such that v[i]=l if l copies of region rᵢ are present in the configuration and v[i]=0 if no such copy is present. A single copy of CYP2D6, for the case when no structural variations are present, will be represented as a vector v consisting of n ones followed by n zeros. Analogously, this applies to a single copy of CYP2D7 (zeros followed by ones). As long as the gene of interest has dwell established pseudogenes with which it can hybridize, vector v will have n(d+1) dimensions.

An aim of some embodiments is to first derive the aggregate copy number of each exon and intron of the gene from read mappings. Then, for each possible configuration that could be present in the sample, the corresponding vector v is constructed. “V={v₁, v₂, . . . , vₖ}” denotes the set of all such vectors. Any vector v for which each of its dimensions is upper bounded by the aggregate copy number of the corresponding exon or intron, is a possible candidate vector to be included in the set V. However, since there are exponentially many such vectors, inclusion is restricted to vectors that correspond to hybrid configurations described in the gene reference database(s).

In some embodiments, the number of whole copies of CYP2D6 and CYP2D7 is determined, as is the number of copies of each hybrid gene and each structural variation (more specifically, each partial gene deletion and partial gene duplication) configuration described in the database. In some embodiments, this is achieved by determining the set of configurations which, collectively, match the aggregate copy number profile of each intron and exon, as closely as possible.

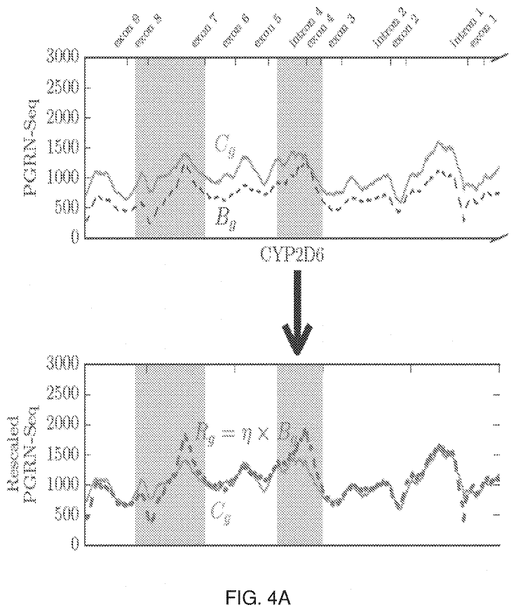

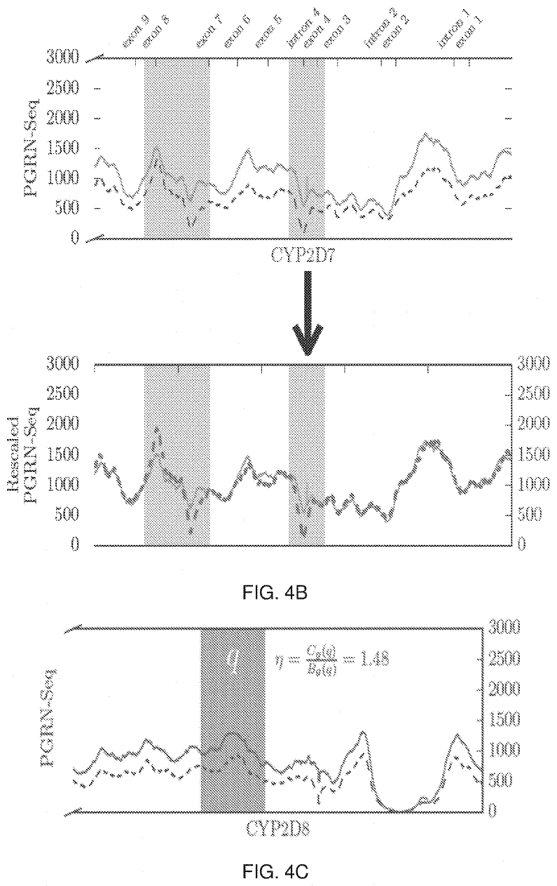

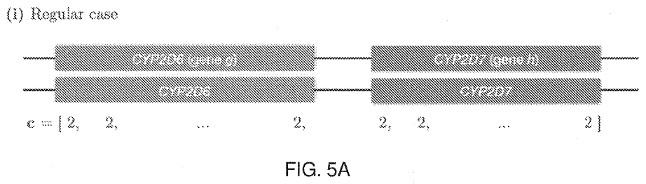

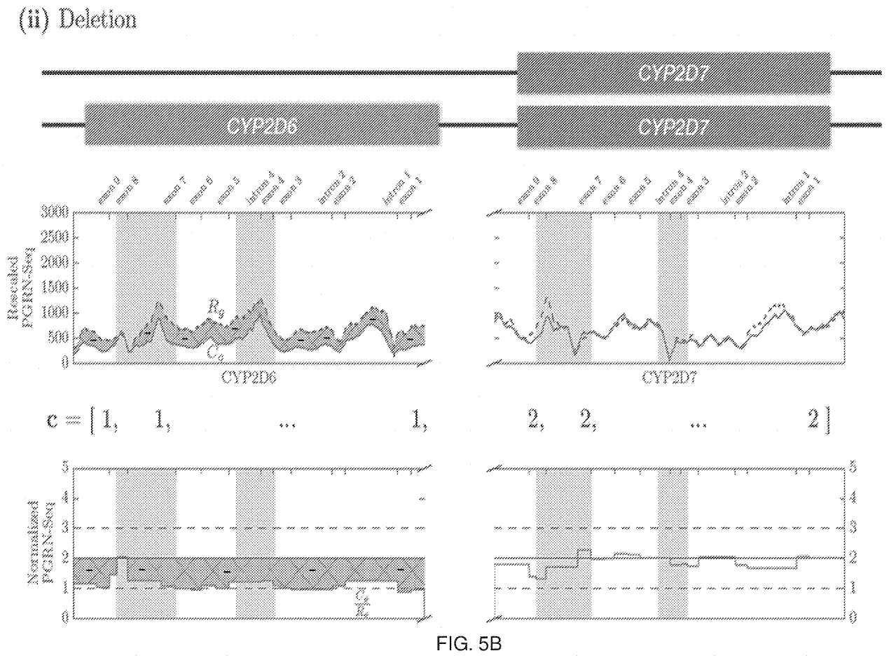

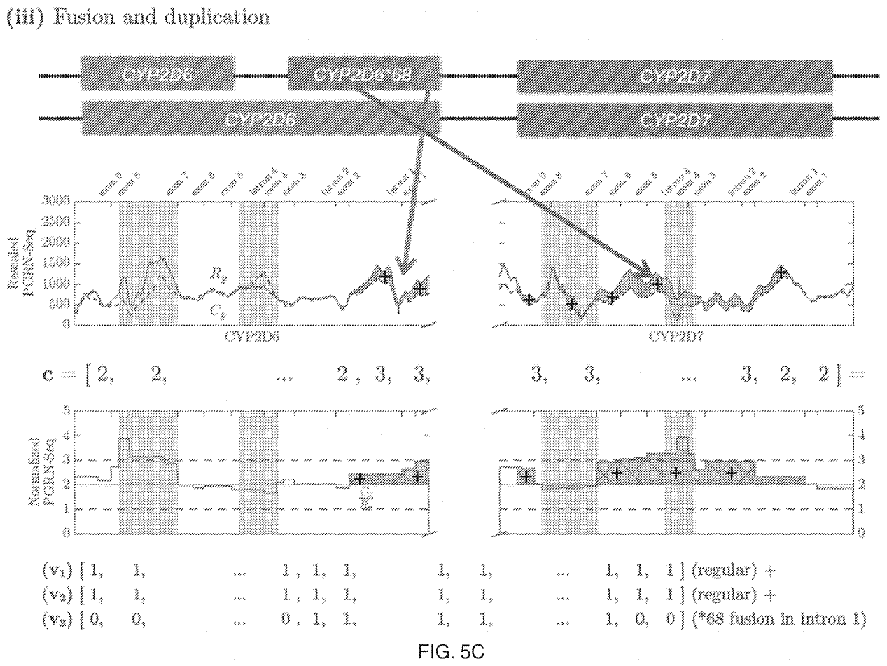

In some embodiments, to estimate the copy number of any region r spanning positions a, a+1, . . . , b of a gene or pseudogene s, the normalized copy number cnₛ of s is calculated first, which reflects the number of copies of s at position i (when the intron/exon of the gene includes ambiguously mappable positions, those positions are ignored). Details about calculating this function are provided in the section “Coverage Normalization” and FIGS. 4 and 5. The estimated copy number (or observed coverage) of a region r of s is denoted as cn[r], and is calculated as:

$$cn [ r ] = \frac{{\sum }_{a \leq i \leq b} {cn}_{s} ( i )}{b - a + 1} .$$

The goal is then formulated as an instance of integer linear programming (ILP), where the goal is to find an integer linear combination Σᵢ₌₁^{k} zᵢvᵢ of configuration vectors from the set V such that the sum

$${\sum }_{r ∈ R} \vert {G}_{r} \vert$$

$$where$$

$${G}_{r} = cn [ r ] - {\sum }_{i = 1}^{k} {z}_{i} {v}_{i} [ r ] foreachr ∈ R$$

is minimized. Non-negative integer variables zᵢ denote the number of times (i.e., the copy number of) a configuration described by vector vᵢ from the database appears in the solution. k denotes the total number of possible configurations. This is termed the “Copy Number Estimation Problem” (CNEP).

In some embodiments, after finding all optimal solutions of CNEP, only the most parsimonious solutions are reported, i.e., those for which Σᵢ₌₁ zᵢ is the minimum possible. In some embodiments when multiple optimal solutions minimize the term Σᵢ₌₁ zᵢ, all solutions will be reported in the final output of CNEP.

For example, let n=2. Let the aggregate copy number vector be cn=[1111], and the set of potential vectors be V={v₁, v₂, v₃, v₄} where v₁=[1100], v₂=[0011], v₃=[1000], and v₄=[0100]. Consider the following optimal solutions to this instance of CNEP: (z₁, z₂, z₃, z₄)=(1, 1, 0, 0) and (z₁, z₂, z₃, z₄)=(0, 1, 1, 1). The first solution is more parsimonious compared to the second one since the first solution implies the presence of the whole gene and the whole pseudogene—whereas the second solution implies the presence of two structurally altered copies of the gene itself, in addition to the whole pseudogene.

It should be noted that certain regions in CYP2D6 and CYP2D7 are identical (e.g. intron 7 or exon 8). The ADME genotyping methods of the embodiments of the present disclosure do not include these regions in the analysis performed above, because many misaligned reads originating from these regions significantly affect the accurate calculation of cn[r]. Impact of the misaligned reads is clearly visible in FIGS. 5B and 5C, where the identical regions are shaded. There are only a few alleles having breakpoints in these regions. However, in these cases the genotyping methods described will still identify the presence of fusion events and predict the correct phenotype. For example, allele CYP2D6*13G1 with a breakpoint in intron 7 will be detected as CYP2D6*13E, which has a breakpoint in exon 5. Both of these alleles represent a non-functional fusion with CYP2D7.

### Coverage Normalization

While sequencing technologies such as Illumina HiSeq provide data having uniform coverage, coverage using the PGRNseq platform is highly non-uniform across regions and its depth is usually not known in advance. By analyzing 96 samples sequenced using the PGRNseq platform, it was discovered that the depth of coverage generally follows the same shape across different samples, as illustrated in FIGS. 4A-4C.

In some embodiments, in order to characterize the shape of depth of coverage for PGRNseq data for a given ADME gene, data PGRNseq data from a reference sample is used. For example, in order to characterize the shape of depth of coverage for CYP2D6 of an arbitrary target sample S, PGRNseq data from wild-type individual NA19686 can be utilized. The reference sample can be any sample from an individual of known genotype (e.g., wild-type) for the ADME gene of interests. In this case, the reference sample is a cell line from an individual with a CYP2D6 genotype of two reference *1 alleles (wild-type).

In some embodiments, a sum of coverage depth (Bₛ) of both chromosomal copies is calculated for i-th nucleotide of gene s from a reference sample (e.g., NA19686), where the sum of coverage depths is calculated according to sequencing conditions used to determine the sequence of the reference sample. In some embodiments, this is calculated as

Bₛ:{1,2, . . . ,|g|}→R,

where s and |s| denote the gene of interest (e.g., CYP2D6, CYP2D7, or CYP2D8) and its respective length, respectively. The value of Bₛ(i) equals to the sum of coverage depths of both chromosomal copies for i-th nucleotide of gene s in the reference sample.

In some embodiments, sequencing experiments generating data for target sample S and the reference sample are not necessarily equivalent in terms of depth of coverage. Consequently, an appropriate rescaling of function Bₛ is calculated in order to obtain a function of reference coverage depth for the sequencing experiment of sample S. Analogously to Bₛ, the rescaled function Rₛ is defined as

Rₛ:{1,2, . . . ,|g|}→R,

Rₛ(i) represents a depth of coverage for i-th nucleotide of the wild-type sample (i.e., the reference sample) sequenced under the same conditions as target sample S. As Bₛ and Rₛ follow the same shape, Rₛ can be estimated as

Rₛ(i)=η×Bₛ(i),∀i∈{1,2, . . . ,|s|}

where η is the ratio of sequencing depths of the two experiments.

In order to determine η, Bₛ and a depth of observed coverage function for target sample S, here denoted as Cₛ and defined analogously to Rₛ and Bₛ are used. Using a region q of the gene of stable copy number that is known to not be involved in any structural variations, η can be estimated as

$$η = \frac{{C}_{s} ( q )}{{B}_{s} ( q )} ,$$

where Cₛ(q) and Rₛ(q) are obtained by summing all values of Cₛ(i) and Rₛ(i), respectively, for any nucleotide i falling into the region q.

One of the regions from CYP2D locus having this property is the region of CYP2D8 containing exons 4, 5 and 6. Using this region as q in the above formula leads to a suitable estimate of η that can then be used for computing the reference coverage depth function Rₛ for target sample S. In some embodiments, Rₛ and Cₛ are not necessarily identical due to the possible presence of structural variations in target sample S. Examples of PGRNseq coverage rescaling are illustrated in FIGS. 4A-4C. The solid grey line indicates the coverage of target sample S (Cₛ), while the thinner dashed line indicates wild-type sample coverage Bₛ. The thicker dashed line indicates rescaled Rₛ=η× B_{g}. Shading in FIG. 4C denotes the region q from CYP2D8. Identical regions in FIGS. 4A and 4B are shaded.

Due to the existence of rearrangement events, the copy number of regions as large as the whole gene cannot be directly estimated. Namely, in the case of a hybrid gene, it is not possible to define the exact number of CYP2D6 and CYP2D7 whole-gene copies, as only a portion of each gene is involved in the corresponding fusion event. Thus, in some embodiments, the copy number status of smaller gene regions are considered to allow proper characterization and detection of these complicated cases. For this purpose, assume that CYP2D6 and CYP2D7 are split into regions r₁, r₂, . . . , and r′₁, r′₂, . . . , r′ₙ, respectively. Due to high sequence similarity between genes CYP2D6 and CYP2D7, it can be assumed that the breakpoints for two splits are equivalent. In some embodiments, in order to characterize rearrangement configurations, a binary vector v consisting of 2n entries is used, which is defined as follows:

$$v [ i ] = \{ \begin{matrix}0 , & if i \leq n and {r}_{i} is not present in a given configuration \\ 0 , & if i \leq n and {r}_{i}^{′} is not present in a given configuration , \\ 1 , & otherwise .\end{matrix}$$

Considering CYP2D locus at the single chromosome, note that the most frequent case where one copy of each of CYP2D6 and CYP2D7 is present can be simply represented as a vector v having all entries equal to 1. A deletion case can be represented as a vector v of n zeroes followed by n ones, whereas a multiplication case containing k copies of CYP2D6 without hybrid genes can be represented as a sum of [1, 1, . . . , 1] and k−1 vectors [1, 1, . . . , 1, 0, 0, . . . , 0], where the first n entries of the last vector are equal to 1 and the remaining n entries are equal to 0. Some examples considering CYP2D locus on both autosomes and covering some more complicated cases including hybrid genes are illustrated in FIGS. 4A-4C.

FIGS. 5A-5C illustrate examples of PGRNseq coverage normalization for three CYP2D6 gene arrangements. For each case, the first row indicates the rescaled PGRNseq coverage, while the second row indicates normalized coverage (i.e. Cₛ|Rₛ). Regions labeled “−” denote deletion of CYP2D6, while regions labeled “+” indicate duplication. Identical regions are shaded. Example (i) illustrates a normal arrangement having one copy of each of CYP2D6 and CYP2D7 on both chromosomes. Example (ii) illustrates CYP2D6 deletion on one chromosomal copy. Example (iii) illustrates one copy of CYP2D6*1 on one chromosomal copy accompanied by a CYP2D7/2D6 fusion (*68 allele) with the breakpoint in intron 1 on the other chromosomal copy. Note the changes in vector c, which describes each copy number structure. In example (iii), the set of vectors v which most closely describe vector c is given under the figure.

In some embodiments, an optimal gene arrangement in target sample S is identified by determining a minimal difference between the observed coverage of each of the gene regions and coverage formed by one or more known possible arrangements for the gene. In some embodiments, the function cnₛ(i) denotes the normalized copy number at loci I within the gene s as:

$${cn}_{s} ( i ) = 2 \times \frac{{C}_{s} ( i )}{{R}_{s} ( i )}$$

Coefficient 2 is included to account for both autosomes present in the data sets.

Analogously, in some embodiments, the function mutcn(m) denotes the estimated copy number of m. For a mutation m, the value of mutcn(m) is obtained by normalizing the number of reads that include m by the expected coverage of m's locus, which is upper bounded by the normalized copy number of the region that covers m's locus, as it is not necessary that all reads that are mapped to this locus include mutation m, as they may originate from other copies of the gene.

### Major Star-Allele Identification

In some aspects, a goal of genotyping is to accurately predict the gene's final protein product, which is equivalent to the detection of a major star-allele of each gene copy discovered. i.e. i.e. In some embodiments, sets of gene-disrupting mutations and corresponding star-alleles for each ADME gene to be genotyped can be obtained from an ADME gene allele database, or curated from a general genomic database. Representative databases include the Human Cytochrome P450 (CYP) Allele Nomenclature Database (available at cypalleles.ki.se), the Leiden Open Variation Database (available at databases.lovd.nl/shared/genes), which includes variant data for many ADME genes, a gene-specific database such as The Human Cytochrome P450 Allele Nomenclature Database (available at cypalleles.ki.se/), or a general genomic database such as Gene, which is maintained by the National Center for Biotechnology Information (available at ncbi.nlm.nih.gov/gene), although other databases exist and may be similarly used in the identification of both functional and neutral mutations. In some embodiments, the database can be a unique database comprising curated data for one or more ADME genes.

In some embodiments, detection of the major star-allele for each gene copy requires first identifying the set M={m₁, m₂, . . . , m_{w}} of all gene disrupting mutations (as defined by the gene allele database(s)) detected in the sample. All major star-alleles for which at least one of the defining gene-disrupting mutations is not present in M are removed from consideration. Also removed from consideration are all alleles with a structural configuration that is not reported among any of the solutions of CNEP (some major star-alleles of CYP2D6 are hybrid configurations with CYP2D7). A={a₁, a₂, aₜ} denotes the set of major star-alleles remaining after the filtering step, and gdm(a) denotes the set of gene disrupting mutations of allele a.

Non-negative integer variables p₁, p₂, pₜ are utilized, where pₜ represents the number of copies of the major star-allele aᵢ in the genotype reported in the solution. For an arbitrary mutation m:

$${E}_{m} = mutcn ( m ) - {\sum }_{i : m ∈ gdm ( {a}_{i} )} {p}_{i} ,$$

where mutcn(m) denotes the estimated copy number of m. For a mutation m (e.g., of the wild-type CYP2D6), the value of mutcn(m) is obtained by normalizing the number of reads that include m by the expected coverage of m's locus, which is upper bounded by the normalized copy number of the region that covers m's locus, as it is not necessary that all reads that are mapped to this locus include mutation m, as they may originate from other copies of the gene.

In some embodiments, it is assumed that the presence of gene-disrupting mutation m in the target sample implies that its genotype contains at least one of the major star-alleles harboring m. For each m ∈ M the following constraint can be added:

$${\sum }_{i : m ∈ gdm ( {a}_{i} )} {p}_{i} \geq 1.$$

In some embodiments, a set of major star-alleles is selected which most closely matches the observed set M of gene-disrupting mutations. In some embodiments, this is achieved by:

$${\sum }_{m ∈ M} \vert {E}_{m} \vert .$$

This is the Major Star-Allele Identification Problem (MSAIP), which is formulated/solved as an ILP. In some embodiments, if multiple optimal solutions to the MSAIP are obtained, all of them will be processed by a final genome refining step.

In some embodiments, the final genome refining step comprises using pairs of mutation loci that are covered by a single read to eliminate major star-alleles whose mutational compositions contradict the reads covering such mutations. In some embodiments, the filter considers reads that cover the locus of at least two gene-disrupting mutations, and eliminates major star-alleles which do not agree with (i.e., are not in phase with) any of the reads that cover these mutation loci.

In some embodiments, in order to avoid the presence of alleles which contain non-expressed gene-disrupting mutations, it is required that:

Σ_{i:m}_{ⱼ}_{∈P}_{ᵢ}Pᵢ≤0 if if C_{g}(mⱼ)=0.

In some embodiments, this model can produce multiple optimal solutions. Due to the short length of the PGRNseq reads (100 bp) and short insert size (around 300 bp), in some embodiments, this model is not able to precisely resolve cases where one set of distant mutations (i.e. mutations that cannot be spanned by read pairs) describes multiple star-alleles. In some embodiments, such optimal star-alleles will be passed to the genotype refining step described herein, where the final genotype determination will be made.

In some embodiments, no set of star-alleles from the database can satisfy all constraints, making MSAIP infeasible. This indicates that the sample contains major star-alleles which are not present in the database(s). In such instances, an empty set is reported as the output of MSAIP and the genotype refining step described herein is used to construct novel major star-alleles that best described the observed set of mutations. The presence of novel major star-alleles does not necessarily imply infeasibility of MSAIP; as long as the set of observed mutations can be explained with available major star-alleles from the database, those star-alleles will be returned as the output.

### Genotype Refining

Since certain gene-disrupting mutations are present in multiple major star-alleles, several mutations must be used to distinguish such major star-alleles. In cases where subsequent gene-disrupting mutations are “distant” (i.e., farther away than the read/fragment length), and are shared by multiple major star-alleles, it may not be possible to unambiguously identify the specific major star-alleles present in the sample. In such cases, the major star-allele identification step can produce multiple equally plausible solutions that “explain” the set of input reads. In some embodiments, this ambiguity is resolved in that some neutral mutations are more likely to occur within certain major star-alleles. The use of neutral mutations in the genotyping process is termed the Genotype Refining Problem (GRP). In some embodiments, the final allelic decomposition of the sample is established by solving GRP. Because the genotype refining step is highly flexible, the tool and methods described herein are able to assign additional neutral mutations to each star-allele and thus establish novel star-alleles. As a result, the genotype refining step not only allows for the selection of the best major star-allele configuration for the sample, but also to assign each neutral mutation to its major star-allele of origin.

GRP is similar to MSAIP. In some embodiments, the input to GRP is a set of major star-alleles inferred in the major star-allele identification step, and the goal is to “extend” each such major star-allele definition to a minor star-allele definition. As a result, the goal of GRP is to return a collection of minor star-alleles such that each is an extension to a unique major star-allele identified by solving MSAIP as described in the major star allele identification section herein. Let mut(a) denote the set of all (both gene-disrupting and neutral) mutations defining a minor star-allele a. Consider each major star-allele b identified in the major star-allele identification step and each minor star-allele a that is an extension to b (e.g., *2A is an extension of *2). Let binary variable x_{a,b} indicate whether the solution of GRP “identifies” a as the “correct” extension of b. For a given major star-allele b, x_{a,b}=1 for at most one minor star-allele a. In some embodiments, all minor star-alleles in the sample are known/defined in the database. Where all minor star-alleles in the sample are known/defined in the database, the goal of GRP is to find for each major star-allele b identified in the solution of MSAIP, the minor star-allele a for which x_{a,b}=1 based on mut(a). In some embodiments, such a solution to GRP is infeasible. Where a solution to GRP is infeasible, a new minor star-allele a′ is “defined” in the most parsimonious manner so that mut(a) ∩mut(a′) is the maximum possible.

In some embodiments, for each x_{a,b} and for each m∈ mut(a), a binary variable e_{a,b,m} is introduced, which is equal to 1 only if m∈ mut(α′). For each x_{a,b} and for each m ∉ mut(a), a binary variable f_{a,b,m} is also introduced, which is equal to 1 only if m∈ mut(a′). Now all mutations m are considered, either observed in the sample, or are present in the minor allele-descriptions. In some embodiments, the primary goal of GRP is the minimization of the (weighted) difference between f_{a,b,m} and a_{a,b,m} across all mutations m and all minor-major allele pairs a,b such that x_{a,b}=1.

In some embodiments, the goal is to assign each observed mutation m to one or more of the major star-alleles identified in the major star-allele identification step to obtain a possibly new minor star allele a′ for each major star allele b, such that min_{∀a∈database}|mut(a′)∩^{mut}(a)| is minimized across all minor star-alleles a′ in the solution of GRP. In some embodiments, this is achieved by ensuring for each mutation m that the number of minor star-alleles a′ in the solution to GRP for which m ∈ mut(a′) is as close as possible to mutcn(m), the estimated copy number of m. The difference between mutcn(m) and the number of minor star-alleles in the solution that includes m can be expressed as:

$${F}_{m} = mutcn ( m ) - [ {\sum }_{( a , b ) : m ∈ mut ( a )} {x}_{a , b} {e}_{a , b , m} + {\sum }_{( a , b ) : m ∉ mut ( a )} {x}_{a , b , m} ]$$

$$for all mutations m .$$

Thus, in some embodiments, the objective of GRP is to simultaneously minimize Fm as well as the difference between f_{a,b,m} and e_{a,b,m} across all mutations m. These to objectives are combined linearly as:

min{Σₘ|Fm|+Σ_{a,b}x_{a,b}[α(Σ_{m∈mut(a)}(1−e_{a,b,m}))+β(Σm∉mut(a) f_{a,b,m})+η(Σ_{m∈M\mut(a)}f_{a,b,m})]}, (1)

where α and β, respectively, denote the penalty scores for missing and added mutations to the selected minor star-alleles from the database. In some examples presented herein, α=2 and β=1 were used, since the likelihood of a minor star-allele to contain a new mutation was roughly twice that of lacking a known mutation.

In some embodiments, the above objective provides a way to modify a major star-allele description: here η represents the penalty of assigning a gene-disrupting mutation to a minor star-allele (thus modifying its major allele, and thus its functional impact). In some embodiments, the value of η is set very high (currently η=100,000) to make sure that gene-disrupting mutations are allowed to produce novel major star-allele descriptions only if MSAIP produces no valid solutions (implying that a novel major star-allele is present in the sample). e_{a,b,m} is always 1 if m is a gene-disrupting mutation (i.e., gene-disrupting mutations from the minor star-allele is not permitted).

In some embodiments, it can be guaranteed that each major star-allele associated with a minor star-allele in a solution is assigned all of its gene-disrupting mutations. To achieve this, let Dₐ be the set of gene-disrupting mutations from the set mut(a). This requirement can be expressed by the following set of constraints:

$$\begin{matrix}( \vert {D}_{a} \vert - {\sum }_{m ∈ {D}_{a}} {e}_{a , b , m} ) {x}_{a , b} = 0 & for each {a}_{a , b}\end{matrix} .$$

In some embodiments, it can be ensured that no variation is “over-called” (i.e., the copy numbers of each of the minor star-alleles in the solution must be consistent with the estimated copy number of the mutations they include) through the use of additional sentinel constraints.

In some embodiments, GRP requires finding the set of minor star-alleles for which Eq. 1 gives the lowest score. In some embodiments, GRP is solved as a QIP (Quadratic Integer Program), returning the minor star-alleles obtained as the final genotype. In some embodiments, the tool and methods described herein solves GRP for each one of the optimal solutions for CNEP and MSAIP, which GRP takes as input. In some embodiments, all such solutions are refined, and the genotype is identified as that with the lowest scoring objective.

### Complexity

In some embodiments, the Copy Number Estimation Problem (CNEP) and the Major Star-Allele Identification Problem (MSAIP) are NP-hard.

### Systems

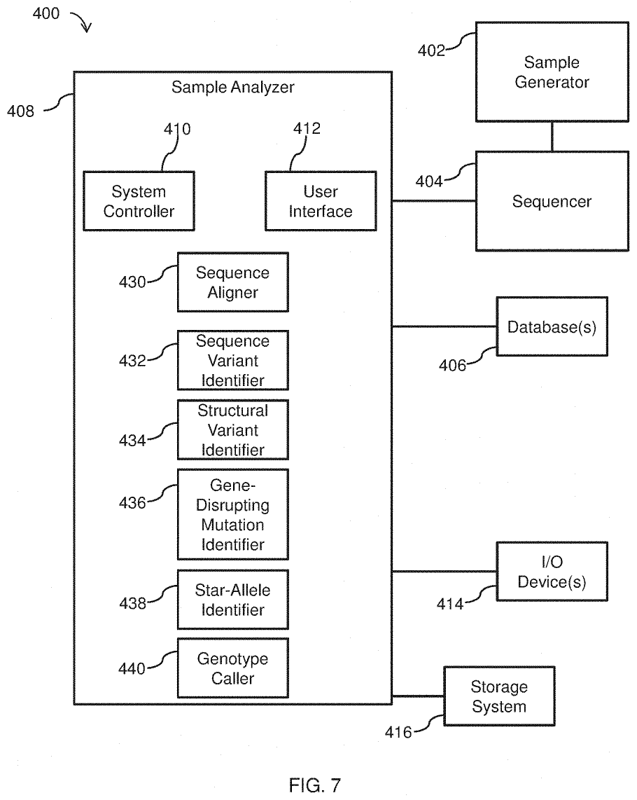

In some embodiments, the methods for predicting genotype described herein are carried out on one or more suitably programmed computers. In some aspects, the methods for predicting genotype described herein are carried out on a genotype predictor system. FIG. 7 illustrates a genotype predictor system 400 formed in accordance with an embodiment that can be used to carry out the methods disclosed and described herein. For example, the system 300 can be used to carry out the methods described herein, such as methods 100 (FIG. 1A), 200 (FIG. 2A), 200′ (FIG. 2B), and 300 (FIG. 3). Various steps of the methods can be automated by the system 400, such as sequencing, analysis, or both, whereas one or more steps may be performed manually or otherwise require user interaction. In some embodiments, the user provides a target sample (e.g., blood, serum, saliva, hair, etc.) and the system 400 automatically prepares, sequences, and analyzes the target sample and provides a genotype profile of the source(s) of the target sample. In other embodiments, the user provides the genotype predictor system 400 instructions to acquire sequence data from a source (e.g., a database or a sequencing facility), to analyze the sequence data, and to provide a genotype profile of the source. In some embodiments, the sequence data is obtained using the PGRNseq platform. In other embodiments, the sequence data is WGS data. In some embodiments, genotype predictor system 400 is an integrated standalone system that is located at one site. In other embodiments, one or more components of the system are located remotely with respect to each other. For example, in some embodiments, the sample generator 402 and sequencer 404, the databases 406, and the sample analyzer 408 may be implemented in multiple instances, distributed across multiple computing devices, instantiated within multiple virtual machines, and the like.

As depicted, the genotype predictor system 400 includes a sample generator 402, a sequencer 404, one or more databases 406, a sequence analyzer 408, one or more input/output (I/O) devices 414, and a storage system 416. In some embodiments, the sample generator 402 prepares a target sample for a designated sequencing protocol such as, for example, PGRNseq or whole genome sequencing (WGS). The sample generator can prepare the target sample according to known methods.

The sequencer 404 then conducts the sequencing according to known protocols, such as Illumina WGS or PGRNseq, to generate the sequencing data. In some embodiments, the sample analyzer 408 receives the sequencing data from the sequencer 404. In other embodiments, the sample analyzer 408 receives the sequencing data from an intermediate source, such a database.

FIG. 7 provides a block diagram of a sample analyzer 408 formed according to one embodiment. In some embodiments, the sample analyzer 408 is used to analyze high throughput sequencing (HTS) data to prove a genotype call for one or more genes (e.g., ADME genes) and generate a genetic profile of a target sample. In some embodiments, the sample analyzer 408 includes a system controller 410 and a user interface 412. The system controller 410 is communicatively coupled to the user interface 412 and/or the sample generator 402 and sequencer 404. In some embodiments, the system controller 410 includes one or more processors/modules to process and, optionally, analyze the HTS data in accordance with the methods described herein. For example, in some embodiments, the system controller 410 includes one or more hardware, firmware, and/or software modules (or a combination thereof) each configured to execute a set of instructions that are stored in one or more volatile or non-volatile storage elements (e.g., instructions stored on a tangible and/or non-transitory computer readable storage medium) to process the HTS data. In some embodiments, the set of instructions includes various commands that instruct the system controller 410 as a processing machine to perform specific operations such as the workflows, processes, and methods described herein.

As illustrated, the system controller 410 includes a plurality of modules or sub-modules that control operation of the system controller 410. These sub-modules may be included in the same device or distributed among multiple devices and/or across multiple networked devices. In some embodiments, the system controller 410 includes modules 430-440 and is connected to a storage system 416 and one or more databases 406. The storage system 416 and databases 406 can communicate with at least some of the modules 430-440. In some embodiments, the modules include a sequence aligner 430, a sequence variant identifier 432, a structural variant identifier 434, a gene-disrupting mutation identifier 436, a star-allele identifier 438, and a genotype caller 440. In some embodiments, the genotype predictor system 400 includes other modules or sub-modules of the modules, configured to perform the operations and methods described herein.

The sequence aligner 430 is configured to receive the HTS data from the target sample and a database 406 of a reference sequence, and to align the HTS data from the target sample to the reference sequence according the methods described herein under the heading Alignment.

The sequence variant identifier 432 is configured to identify and call nucleic acid sequence variation in the target sample relative to the reference sequence according to the methods described herein under the heading Sequence Variant Calling. In some embodiments, the sequence variant identifier 432 receives information about known gene sequence variants via a database.

The structural variant identifier 434 is configured to identify structural variants of genes included in the HTS data from the target sample according to the methods described herein under the heading Detecting Structural Variants.

The gene-disrupting mutation identifier 436 is configured to identify which of the variants detected by the sequence variant identifier 432 and/or 434 results in a gene-disrupting mutation according to the methods described herein under the heading Major Star-Allele Identification. In some embodiments, the gene-disrupting mutation identifier 436 receives information regarding known gene-disrupting mutations for one or more genes via one or more databases.

The star-allele identifier 438 is configured to identify one or more star-alleles of a reference gene that has the identified gene-disrupting mutation according to the methods described herein under the heading Major Star-Allele Identification. In some embodiments, the star-allele identifier 338 receives information for known gene alleles for one or more genes via one or more databases.

The genotype caller 440 is configured to determine the genotype of each target gene of the HTS data from the target sample according to the methods described herein under the heading Major Star-Allele Identification. In some embodiments, the genotype caller includes a genotype refiner 440′ configured to rank each possible genotype called by the genotype caller 440 and to identify one or more genotypes with the best ranking score, as described herein.

The modules 430-440 are configured to carry out the methods as described herein.

By way of example, the sample analyzer 408 can be or include a desktop computer, a laptop computer, a notebook computer, a tablet computer, or a smart phone. In some embodiments the user interface 412 includes hardware, firmware, software, or a combination thereof that enables a user to directly or indirectly control operation of the system controller 410 and at least some of the various components thereof. In some embodiments, the system 400 includes an input/output (I/O) device(s) 414, such as a keyboard, display printer, universal serial bus (USB) port, a speaker, pointer device, trackball, button, switch, touch screen, and the like.

In some embodiments, the genotype predictor system 400 displays the genotyping results on an I/O device 414 that is a display. In other embodiments, the genotype predictor system 400 is configured to deliver the genotyping results to a printer, an email address, or other device.

In some embodiments, the genotype predictor system 400 produces a genotype report, providing the genotype for each gene for which HTS data was analyzed. In some embodiments, the genotype report provides flags or notices for those genes of the target sample that vary in genotype and/or phenotype from a known star-allele. In some embodiments, the genotype report also or only provides flags or notices for those genotypes that will affect absorption, distribution, metabolism, or excretion of a pharmaceutical compound.

As used herein, the terms “module,” “system,” and “system controller” can refer to a hardware and/or software system and circuitry that operates to perform one or more functions. A module, system, or system controller may include a computer processor, controller, or other logic-based device that performs operations based on instructions stored on a tangible and non-transitory computer readable storage medium, such as a computer memory. Alternatively, a module, system, or system controller can include a hard-wired device that performs operations based on hard-wired logic and circuitry. The module, system, or system controller depicted in FIG. 7 can represent the hardware and circuitry that operates based on software or hardwired instructions, the software that directs hardware to perform the operations, or a combination thereof. The module, system, or system controller can include or represent hardware circuits or circuitry that include and/or are connected with one or more processors, such as one or more computer microprocessors.

As used herein, the terms “software” and “firmware” are interchangeable, and include any computer program stored in memory for execution by a computer, including Random Access Memory (RAM), Read Only Memory (ROM), Electronically Erasable Programmable Read Only Memory (EEPROM), non-volatile RAM (NVRAM), flash memory, optical or holographic media, magnetic cassettes, magnetic tape, magnetic disk storage or other magnetic storage devices, data transmissions, or any other medium that can be used to store information and can be accessed by a computing device. The above memory types are representative only, and are thus not limiting as to the types of memory usable for storage of a computer program.

In some embodiments, a processing unit, processor, module, or computing system that is “configured to” perform a task or operation can be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). A general purpose computer (which may become “configured to” perform the task or operation if appropriately programmed) is not “configured to” perform a task or operation unless or until specifically programmed or structurally modified to perform the task or operation.

In some embodiments, the memory stores computer-executable instructions for causing the system controller 308 to implement aspects of embodiments of system components discussed herein and/or to perform aspects of embodiments of methods and procedures discussed herein. Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, elements of the genotype predictor system 400, such as the sequencer 404 (or intermediary HTS data source), database(s) 406, sequence analyzer 408, system controller 410, and user interface 412, are communicatively coupled by one or more communication links. In some embodiments, the one or more communication links can be, or include, a wired communication link such as a USB link, a proprietary wired protocol, and the like. The one or more communication links can be, or include, a wireless communication link such as a short-range radio link, such as Bluetooth IEEE 802.11, a proprietary wireless protocol, and the like.

The term “communication link” can refer to an ability to communicate some type of information in at least one direction between at least two elements of a computer system, and should not be understood to be limited to a direct, persistent, or otherwise limited communication channel That is, according to some embodiments, the communication link may be a persistent communication link, an intermittent communication link, an ad-hoc communication link, and the like. The communication link can refer to direct communications between the sequencer 404 (or intermediary HTS data source) and the sample analyzer 408, between the database(s) 406 and the sample analyzer 408, between the user interface 412 and the sample analyzer 408, or any other combination of the elements of the 4 genotype predictor system 400. The communication link can also refer to indirect communications that travel between the sequencer 404 (or intermediary HTS data source) and the sample analyzer 408, between the database(s) 406 and the sample analyzer 408, between the user interface 412 and the sample analyzer 408, or any other combination of the elements of the 4 genotype predictor system 400, wherein the indirect communication occurs via at least one other device (e.g., a repeater, router, hub, and/or the like). The communication link can facilitate uni-directional and/or bi-directional communication between the various elements of the genotype predictor system. In some embodiments, the communication link is, includes, or is included in a wired network, a wireless network, or a combination of wired and wireless networks. Illustrative networks include any number of different types of communication networks such as, a short messaging service (SMS), a local area network (LAN), a wireless LAN (WLAN), a wide area network (WAN), the Internet, a peer-to-peer (P2P) network, or other suitable networks. The network may include a combination of multiple networks. In some embodiments, for example, the genotype predictor system is accessible via the Internet (e.g., the genotype predictor system may facilitate a web-based genotyping service), and a user may transmit one or more HTS data samples including gene sequencing data to the genotype predictor system to predict a genotype profile.

In some embodiments, the system controller 410 causes the sample analyzer 408 to access the HTS data for the target sample from the sequencer 404 (or intermediary HTS data source) and/or the database(s) 406 via a communication link. The intermediary HTS data source storing data from the sequencer 404 and/or the database(s) 406 can be web-based, cloud based, or local. In some embodiments the HTS data and/or the databases 406 are retrieved from a third party, produced by the user, or some combination thereof. The databases 406 can be any collection of information providing, for example, information regarding ADME gene alleles, such as sequences, sequence variants, structural variant, gene-disrupting mutation information, gene product (i.e., protein) information, and the like.

### Examples

The materials, methods, and embodiments described herein are further defined in the following Examples. Certain embodiments are defined in the Examples herein. It should be understood that these Examples, while indicating certain embodiments, are given by way of illustration only. From the disclosure herein and the Examples, one skilled in the art can ascertain the essential characteristics of this disclosure, and without departing from the spirit and scope thereof, can make various changes and modifications to the disclosure to adapt it to various usages and conditions.

Three data sets were used to evaluate the performance of the genotyping tool and methods described herein.

1) 96 new Coriell cell line samples spanning 32 different family trios and multiple ethnic backgrounds. All cell lines were sequenced on the PGRNseq v2 platform and sequenced with Illumina HiSeq to an average coverage of 600×. Genotypes of the sequenced samples contained various CYP2D6 alleles, including those with multiple types of structural variation. CYP2D6 genotypes of all samples were validated with PCR-based genotyping panels. The performance of the genotyping methods described herein is summarized in Table 2. For 11 of these samples, validation of 12 additional ADME genes by PCR-based genotyping was performed. The performance of the ADME genotyping methods described herein for these genes is provided in Table 4. Genotypes of nine additional ADME genes were available for 17 of the samples. This subset was used to evaluate genotyping performance on those additional genes.

TABLE 2
Performance of ADME genotyping methods for CYP2D6 on 96 PGRNseq samples.

| Sample ID | Family | Ethnicity | Gender | CYP2D6 genotype | Genotype prediction |
|---|---|---|---|---|---|
| HG00421 | SH007 | Chinese | F | *2/*10 × N | ✓ (*2/*10 + *10) |
| HG00422 | SH007 | Chinese | M | *2/*10 | ✓ (*2/*10) |
| HG00423 | SH007 | Chinese | C | *10/*10 × N | ✓ (*10/*10 + *10) |
| HG00463 | SH021 | Chinese | F | *36 + *10/*36 + *10 | ✓ (*36 + *10/*36 + *10) |
| HG00464 | SH021 | Chinese | M | *1/*36 + *10 | ✓ (*1/*36 + *10) |
| HG00465 | SH021 | Chinese | C | *36 + *10/*36 + *10 | ✓ (*36 + *10/*36 + *10) |
| HG00592 | SH057 | Chinese | F | *1/*10 | ✓ (*1/*10) |
| HG00593 | SH057 | Chinese | M | *2/*36 + *10 | ✓ (*2/*36 + *10) |
| HG00594 | SH057 | Chinese | C | *1/*2 | ✓ (*1/*2) |
| HG01060 | PR14 | Puerto Rican | F | *1/*41 | ✓ (*1/*41) |
| HG01061 | PR14 | Puerto Rican | M | *1/*4 | ✓ (*1/*4) |
| HG01062 | PR14 | Puerto Rican | C | *1/*4 | ✓ (*1/*4) |
| HG01190 | PR40 | Puerto Rican | F | *68 + *4/*5 | ✓ (*68 + *4/*5) |
| HG01191 | PR40 | Puerto Rican | M | *2/*41 | ✓ (*2/*41) |
| HG01192 | PR40 | Puerto Rican | C | *5/*41 | ✓ (*5/*41) |
| HG01979 | PEL027 | Peruvian | F | *2/*68 + *4 | ✓ (*2/*68 + *4) |
| HG01980 | PEL027 | Peruvian | M | *1/*2 | ✓ (*1/*2) |
| HG01981 | PEL027 | Peruvian | C | *1/*2 | ✓ (*1/*2) |
| HG02259 | PEL042 | Peruvian | F | *1/*2 | ✓ (*1/*2) |
| HG02260 | PEL042 | Peruvian | M | *1/*1 | ✓ (*1/*1) |
| HG02261 | PEL042 | Peruvian | C | *1/*2 | ✓ (*1/*2) |
| NA06984 | 1328 | European | F | *68 + *4/*4 | ✓ (*68 + *4/*4) |
| NA06989 | 1328 | European | M | *9/*9 | ✓ (*9/*9) |
| NA12331 | 1328 | European | C | *4/*9 | ✓ (*4/*9) |
| NA07357 | 1345 | European | F | *1/*6 | ✓ (*1/*6) |
| NA07345 | 1345 | European | M | *1/*1 | ✓ (*1/*1) |
| NA07348 | 1345 | European | C | *1/*6 | ✓ (*1/*6) |
| NA10853 | 1349 | European | F | *2/*41 | ✓ (*2/*41) |
| NA10854 | 1349 | European | M | *1/*4 | ✓ (*1/*4) |
| NA11834 | 1349 | European | C | *2/*4 | ✓ (*2/*4) |
| NA10860 | 1362 | European | F | *1/*4 | ✓ (*1/*4 + *4N) |
| NA10861 | 1362 | European | M | *4/*2 | ✓ (*4/*35) case (1) |
| NA11984 | 1362 | European | C | *1/*2 | ✓ (*1/*35) case (1) |
| NA11891 | 1377 | European | F | *1/*1 | ✓ (*1/*1) |
| NA11892 | 1377 | European | M | *6/*41 | ✓ (*6/*41) |
| NA10865 | 1377 | European | C | *1/*41 | ✓ (*1/*41) |
| NA12003 | 1420 | European | F | *4/*2 or *4/*35 | ✓ (*4/*35) case (1) |
| NA12004 | 1420 | European | M | *2/*41 | ✓ (*2/*41) |
| NA10838 | 1420 | European | C | *2/*4 | ✓ (*2/*4) |
| NA12155 | 1408 | European | F | *1/*5 | ✓ (*1/*5) |
| NA12156 | 1408 | European | M | *1/*4 | ✓ (*1/*4) |
| NA10831 | 1408 | European | C | *4/*5 | ✓ (*4/*5) |
| NA12272 | 1418 | European | F | *1/*1 | ✓ (*1/*1) |
| NA12273 | 1418 | European | M | *1/*1 | ✓ (*1/*1) |
| NA10837 | 1418 | European | C | *1/*1 | ✓ (*1/*1) |
| NA12342 | 1330 | European | F | *4/*41 | ✓ (*4/*41) |
| NA12343 | 1330 | European | M | *1/*5 | ✓ (*1/*5) |
| NA12336 | 1330 | European | C | *5/*41 | ✓ (*5/*41) |
| NA12399 | 1354 | European | F | *1/*1 | ✓ (*1/*1) |
| NA12400 | 1354 | European | M | *1/*68 + *4 | ✓ (*1/*68 + *4) |
| NA12386 | 1354 | European | C | *1/*1 | ✓ (*1/*1) |
| NA12750 | 1444 | European | F | *2/*2 | ✓ (*2/*2) |
| NA12751 | 1444 | European | M | *1/*2 | ✓ (*1/*2) |
| NA12740 | 1444 | European | C | *1/*2 | ✓ (*1/*2) |
| NA12801 | 1454 | European | F | *4/*6 | ✓ (*4/*6) |
| NA12802 | 1454 | European | M | *2/*41 | ✓ (*2/*41) |
| NA12805 | 1454 | European | C | *2/*4 | ✓ (*2/*4) |
| NA12891 | 1463 | European | F | *68 + *4/*41 | ✓ (*68 + *4/*41) |
| NA12892 | 1463 | European | M | *2/*3 | ✓ (*2/*3) |
| NA12878 | 1463 | European | C | *3/68 + *4 | ✓ (*3/*68 + *4) |
| NA18507 | Y009 | Yoruban | F | *2/*4 × N | ✓ (*2/*4 + *4) |
| NA18508 | Y009 | Yoruban | M | *2/*5 | ✓ (*2/*5) |
| NA18506 | Y009 | Yoruban | C | *2/*5 | ✓ (*2/*5) |
| NA18516 | Y013 | Yoruban | F | *1/*17 | ✓ (*1/*17) |
| NA18517 | Y013 | Yoruban | M | *5/*10 | ✓ (*5/*10) |
| NA18515 | Y013 | Yoruban | C | *1/*10 | ✓ (*1/*10) |
| NA19128 | Y077 | Yoruban | F | *17/*17 | ✓ (*17/*17) |
| NA19127 | Y077 | Yoruban | M | *2/*17 | ✓ (*2/*17) |
| NA19129 | Y077 | Yoruban | C | *17/*17 | ✓ (*17/*17) |
| NA19200 | Y045 | Yoruban | F | (*76) + *1/*5 or | ✓ (*1/*5) case (2) |
| | | | | *1/*5 | |
| NA19201 | Y045 | Yoruban | M | *1/*17 | ✓ (*1/*17) |
| NA19202 | Y045 | Yoruban | C | (*76) + *1/*1 | ✓ (*1/*1) case (2) |
| NA19239 | Y117 | Yoruban | F | *13-like?/*17 or | ✓ (*15/*17) case (3) |
| | | | | *15/*17 | |
| NA19238 | Y117 | Yoruban | M | *1/*17 | ✓ (*1/*17) |
| NA19240 | Y117 | Yoruban | C | *13-like?/*17 | ✓ (*15/*17) case (3) |
| NA19685 | M011 | Mexican-Am | F | *1/*2 × 2 | ✓ (*1/*2 + *2) |
| NA19684 | M011 | Mexican-Am | M | *1/*4 | ✓ (*1/*4) |
| NA19686 | M011 | Mexican-Am | C | *1/*1 | ✓ (*1/*1) |
| NA19771 | M031 | Mexican-Am | F | *2/*4 | ✓ (*2/*4) |
| NA19770 | M031 | Mexican-Am | M | *1/*2 | ✓ (*1/*2) |
| NA19772 | M031 | Mexican-Am | C | *2/*4 | ✓ (*2/*4) |
| NA19789 | M037 | Mexican Am | F | *1/*1 | ✓ (*1/*1) |
| NA19788 | M037 | Mexican Am | M | *2/*78 + *2 | ✓ (*2/*78 + *2) |
| NA19790 | M037 | Mexican Am | C | *1/*78 + *2 | ✓ (*2/*78 + *2) |
| NA19700 | 2367 | AA | F | *4/*29 | ✓ (*4/*29) |
| NA19701 | 2367 | AA | M | *1/*17 | ✓ (*1/*17) |
| NA19702 | 2367 | AA | C | *4/*17 | ✓ (*4/*17) |
| NA19818 | 2418 | AA | F | *1/*17 | ✓ (*1/*17) |
| NA19819 | 2418 | AA | M | *2/*4 × 2 | ✓ (*2/*4 + *4) |
| NA19828 | 2418 | AA | C | *2/*17 | ✓ (*2/*17) |
| NA19834 | 2424 | AA | F | *2/*2 | ✓ (*2/*45) case (4) |
| NA19835 | 2424 | AA | M | *1/*2 | ✓ (*1/*45) case (4) |
| NA19836 | 2424 | AA | C | *1/*2 | ✓ (*1/*45) case (4) |
| NA19900 | 2425 | AA | F | *3/*29 | ✓ (*3/*29) |
| NA19901 | 2425 | AA | M | *1/*1 | ✓ (*1/*1) |
| NA19902 | 2425 | AA | C | *1/*29 | ✓ (*1/*29) |

F = father;
M = mother;
C = child

2) The ADME genotyping methods described herein were also tested on 137 cell line samples sequenced on PGRNseq v1 platform with an average coverage of 600×. The genotypes of these samples for 10 key ADME genes were inferred by the methods described herein and were compared with PCR-based genotyping panels. The comparative performance of the present methods on those samples is summarized in Table 5.

3) In addition to the PGRNseq samples, the ADME genotyping methods described herein were also tested on the samples from the Platinum Genome project, which includes 17 individuals from CEPH 1463 family. All those samples were sequenced with Illumina HiSeq 2000 WGS sequencer with the average coverage of 50×. Illumina WGS samples from 1000 Genome project with the coverage exceeding 20× having available validations were also included. Predictions made by the genotyping methods described herein on Illumina samples are summarized in Table 3.

### DISCUSSION

As illustrated in Table 2, the predictions by ADME genotyping methods described herein matched the validated genotypes in most of the cases. Although some discrepancies between the predictions were identified, further investigation revealed that the genotyping panes either made ambiguous or incorrect calls.

For case (1) (see Table 2), *35 allele was called as *2 for the samples NA10861, NA11984 and NA12003. However, as reported by Pratt, V. M., et al. ((2016) J Mol Diagn, 18(1):109-123), NA12003 actually contains *35 allele instead of *2, and this discrepancy is mostly due to the inability of TaqMan assays to properly genotype *35 allele (see Riffel, A. K., et al (2015) Genotyping, 6:312). A similar occurrence was observed in case (4) with samples NA19834, NA19835 and NA19836, with SNP c.1716 G>A. This SNP differentiates alleles *2 and *45, and it is not present in TaqMan assays (see Fang, H. et al. (2014) Pharmacogenomics J, 14(6):564-572).

Case (4) presented *15, another allele problematic for most of the current genotyping platforms. For example, TaqMan assays often confuse it with other alleles (see Riffel, A. K., et al (2015) Genotyping, 6:312). One of the reasons for this is c.137 insT, which defines *15 allele but is also present in all *13 fusion alleles. However, Pratt, V. M., et al. ((2016) J Mol Diagn, 18(1):109-123) confirmed that NA19239's actual genotype contains *15, which matches the present results as well.

In case (2), copy number results for *13-like fusion allele *76 were not clear for samples NA19200 and NA19202. However, additional validation by Fang, H. et al. ((2014) Pharmacogenomics J, 14(6):564-572) confirmed that *76 is not present, which matches the prediction by the present methods. Furthermore, no evidence of increased coverage in CYP2D7 region was found that would be suggested by the existence of *76. These cases indicated that the present methods provided more accurate genotyping results than currently used genotyping panels, especially in the presence of recently discovered alleles. In the case of NA10860, Cypiripi detected *4 allele duplication, which PCR-based methods miss this duplication. Prediction by the present methods was cross-validated by running the present methods on Illumina HiSeq X WGS NA10860 sample publicly available from export.uppmax.uu.se/a2009002/opendata/HiSeqX_CEPH/. This sample was sequenced with approximately 28× depth of coverage (or 14× per chromosome). By simple coverage analysis, it was evident that there were at least 3 copies of CYP2D6, since average coverage of CYP2D6 region was 42×. Thus, it was assumed that the correct allele was indeed *1/*4+*4.

TABLE 3
CYP2D6 genotype inferred by ADME genotyping methods described herein on a
set of 21 publicly available Illumina WGS samples from three “families.”

| Sample ID | Family | Ethnicity | Gender | CYP2D6 genotype | Genotype prediction |
|---|---|---|---|---|---|
| NA19239 | Y117 | Yoruban | F | *15/*17 | ✓ (*15/*17) case (3⁾ |
| NA19238 | Y117 | Yoruban | M | *1/*17 | ✓ (*1/*17) |
| NA19240 | Y117 | Yoruban | C | *15/*17 | ✓ (*15/*17) case (3) |
| NA19900 | 2425 | AA | F | *3/*29 | ✓ (*3/*29) |
| NA12889 | 1463 | European | GF | *4/*41 | ✓ (*4/*41) |
| NA12890 | 1463 | European | GM | N/A | *68 + *4/*68 + *4 |
| NA12891 | 1463 | European | GF | *41/*68 + *4 | ✓ (*41/*68 + *4) |
| NA12892 | 1463 | European | GM | *2/*3 | ✓ (*2/*3) |
| NA12877 | 1463 | European | F | *4/*68 + *4 | ✓ (*4/*68 + *4) |
| NA12878 | 1463 | European | M | *3/*68 + *4 | ✓ (*3/*68 + *4) |
| NA12879 | 1463 | European | C | N/A | *3/*68 + *4 |
| NA12880 | 1463 | European | C | N/A | *68 + *4/*68 + *4 |
| NA12881 | 1463 | European | C | N/A | *68 + *4/*68 + *4 |
| NA12882 | 1463 | European | C | *4/*68 + *4 | ✓ (*4/*68 + *4) |
| NA12883 | 1463 | European | C | N/A | *3/*68 + *4 |
| NA12884 | 1463 | European | C | N/A | *4/*68 + *4 |
| NA12885 | 1463 | European | C | N/A | *68 + *4/*68 + *4 |
| NA12886 | 1463 | European | C | N/A | *3/*4 |
| NA12887 | 1463 | European | C | N/A | *4/*68 + *4 |
| NA12888 | 1463 | European | C | N/A | *4/*68 + *4 |
| NA12893 | 1463 | European | C | N/A | *3/*4 |

Data is from a mother, father, and child from a Yoruban family, an African American individual, and a larger European/CEPH family from Utah. Family relationships are indicated as (G)F/M = (grand)father/mother; C = child.
The samples with a checkmark are those which have also been sequenced by PGRNseq v2 platform. Their CYP2D6 status based on analysis of the PGRNseq v2 data by the methods of the present disclosure is provided in Table 2. CYP2D6 status predictions on WGS data perfectly agree with its predictions based on PGRNseq v2 data as well as the independent validation data. The remaining samples do not have PGRNseq v2 data or an independent validation and thus are marked with N/A. However, the present method's CYP2D6 predictions on these individuals match Mendelian laws of inheritance.

TABLE 4
Genotyping results using the present methods on a set of 11 PGRNseq v2 samples.

| Sample | | | | CYP2A6 | CYP2A6 | CYP2C19 | CYP2C8 |
|---|---|---|---|---|---|---|---|
| ID | Family | Ethnicity | Gender | genotype | prediction | prediction | prediction |
| HG01190 | PR40 | PR | F | *1/*1 | ✓ *1/*1 | ✓ *1/*2 | ✓ *1/*3 |
| NA07357 | 1345 | E | F | *1/*1 | ✓ *1/*1 | ✓ *2/*17 | ✓ *1/*4 |
| NA07348 | 1345 | E | C | *1/*1 | ✓ *1/*1 | ✓ *2/*17 | ✓ *1/*4 |
| NA10854 | 1349 | E | M | *1/*1 | ✓ *1/*1 | ✓ *1/*1 | ✓ *3/*3 |
| NA12003 | 1420 | E | F | *1/*1 | ✓ *1/*1 | ✓ *1/*1 | ✓ *1/*3 |
| | | | | or *1/*8 | | | |
| NA12156 | 1408 | E | M | *1/*1 | ✓ *1/*1 | ✓ *1/*1 | ✓ *1/*1 |
| NA10831 | 1408 | E | C | *1/*2 | ✓ *1/*2 | ✓ *1/*17 | ✓ *1/*4 |
| NA12878 | 1463 | E | C | *1/*1 | ✓ *1/*1 | ✓ *1/*2 | ✓ *1/*3 |
| NA19239 | Y117 | Y | F | *1/*17 | ✓ *1/*17 | ✓ *13/17 | ✓ *1/*2 |
| NA19789 | M037 | MA | F | *1/*1 | ✓ *1/*1 | ✓ *1/*1 | ✓ *1/*3 |
| NA19819 | 2418 | AA | M | *1/*1 | ✓ *1/*1 | ✓ *1/*17 | ✓ *1/*2 |

| Sample | CYP2C9 | CYP3A4 | CYP3A5 | CYP4F2 | TPMP | DPYD |
|---|---|---|---|---|---|---|
| ID | prediction | prediction | prediction | prediction | prediction | prediction |
| HG01190 | ✓ *1/*2 | ✓ *1/*1 | ✓ *1/*1 | ✓ *1/*3 | ✓ *1/*1 | ✓ *1/*9 |
| NA07357 | ✓ *1/*1 | ✓ *1/*1 | ✓ *3/*3 | ✓ *1/*1 | ✓ *1/*1 | [symbol] |
| NA07348 | ✓ *1/*1 | ✓ *1/*1 | ✓ *3/*3 | ✓ *1/*1 | ✓ *1/*1 | \hat{ }✓ *6/*9 |
| NA10854 | ✓ *2/*2 | ✓ *1/*1 | ✓ *1/*3 | ✓ *1/*3 | ✓ *1/*1 | ✓ *1/*1 |
| NA12003 | ✓ *1/*2 | ✓ *1/*1 | ✓ *1/*3 | ✓ *1/*1 | ✓ *1/*1 | ✓ *1/*1 |
| NA12156 | ✓ *1/*2 | ✓ *1/*1 | ✓ *3/*3 | %✓ *2/*3 | ✓ *1/*1 | \hat{ }✓ *1/*5 |
| NA10831 | ✓ *1/*2 | ✓ *1/*1 | ✓ *3/*3 | ✓ *1/*1 | ✓ *1/*1 | \hat{ }✓ *5/*9 |
| NA12878 | ✓ *1/*2 | ✓ *1/*1 | ✓ *3/*3 | ✓ *1/*1 | ✓ *1/*1 | \hat{ }✓ *4/*5 |
| NA19239 | ✓ *1/*1 | ✓ *1/*1 | ✓ *1/*1 | ✓ *1/*1 | ✓ *1/*1 | ✓ *1/*9 |
| NA19789 | ✓ *1/*2 | ✓ *1/*1 | ✓ *3/*3 | %✓ *2/*3 | ✓ *1/*1 | \hat{ }✓ *1/*5 |
| NA19819 | ✓ *1/*1 | ✓ *1/*1 | ✓ *3/*6 | %✓ *2/*3 | ✓ *1/*1 | \hat{ }✓ *5/*9 |

M = mother; F = father; C = child.
PR = Puerto Rican;
E = European;
Y = Yoruban;
MA = Mexican American;
AA = African American
Concordance is indicated by ✓: matches panel validation.
Improvement is indicated by \hat{ }✓: improvements over panel validations.
Failure is indicated by [symbol] : present methods did not identify optimal genotype.
Samples where the present methods identified multiple optimal solutions are indicated by %✓.

No Mendelian inconsistencies were observed when using the ADME genotyping methods described herein on PGRNseq data. This is in sharp contrast with previous PGRNseq data analysis, which relied on SNP callers to infer genotypes (see Gordon, A. S., et al. (April 2016) Pharmacogenet Genomics 26(4):161-168 (Epub January 2016)).

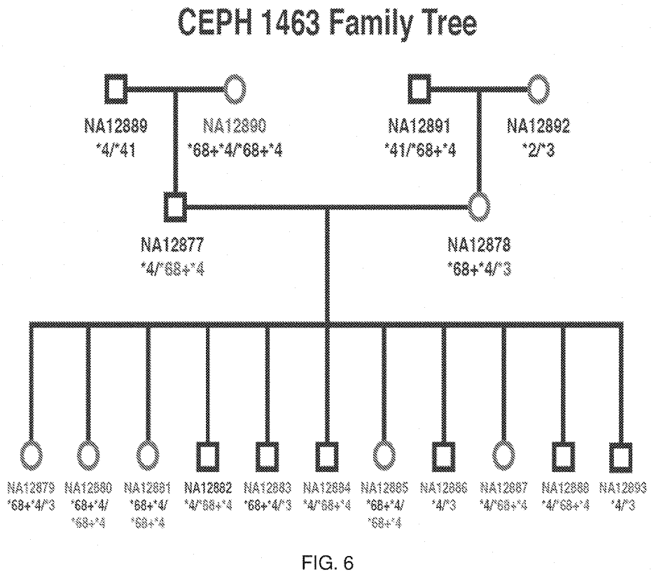

With the Illumina WGS data analyzed, genotypes predicted by the present methods were in concordance with genotypes validated in Twist, G. P., et al., (January 2016) npj Genomic Medicine 1:15007, Fang, H. et al. (2014) Pharmacogenomics J, 14(6):564-572, and Pratt, V. M., et al. ((2016) J Mol Diagn, 18(1):109-123, as summarized in Table 2. Although genotype information for some members of the CEPH 1463 family was not available, genotype predictions made by the present methods are in full accordance with Mendelian laws of inheritance, as illustrated by FIG. 6.

Predictions for CYP2A6 genotype on samples for which the validation was available are summarized in Table 4. The present ADME genotyping methods provided an accurate CYP2A6 genotype call for all samples. Genotype validation for these samples is available in Pratt, V. M., et al. ((2016) J Mol Diagn, 18(1):109-123.

In addition to genotyping accuracy, the methods presented and described herein demonstrated very low computational overhead. In the experiments described, each run required less than 10 seconds and fewer than 100 MB of memory, even for the high-coverage PGRNseq samples.

TABLE 5
Genotyping results using the present methods
on a set of 137 PGRNseq v1 samples.

| Sample | CYP2D6 | CYP2A6 | CYP2C19 | CYP2C8 | CYP2C9 |
|---|---|---|---|---|---|
| Concordance | 106 | 110 | 127 | 137 | 124 |
| Improvement | 29 | 19 | 10 0 | 0 | 13 0 |
| Unknown | 1 | 5 | 0 | 0 | 0 |
| Failure | 0 | 0 | 0 | 0 | 3 |
| PGXT101 | ✓ *1/*4 | ✓ *1/*1 | ✓ *1/*17 | ✓ *1/*1 | ✓ *1/*1 |
| PGXT102 | ✓ *1/*2 | ✓ *1/*9 | ✓ *1/*1 | ✓ *1/*3 | \hat{ }✓ *2/*18 |
| PGXT103 | ✓ *2/*35 | ✓ *1/*1 | ✓ *2/*17 | ✓ *1/*4 | ✓ *1/*1 |
| PGXT104 | ✓ *1/*1 | ✓ *1/*1 | ✓ *1/*1 | ✓ *1/*1 | \hat{ }✓ *1/*18 |
| PGXT105 | ✓ *4/*9 | ✓ *1/*1 | ✓ *1/*1 | ✓ *1/*1 | ✓ *1/*1 |
| PGXT106 | ✓ *1/*2 | ★ *1/*35 | ✓ *1/*17 | ✓ *1/*1 | ✓ *1/*1 |
| PGXT107 | ✓ *1/*2 | ✓ *1/*9 | ✓ *1/*1 | ✓ *1/*1 | ✓ *1/*1 |
| PGXT108 | ✓ *1/*4 | ✓ *1/*9 | ✓ *1/*2 | ✓ *1/*1 | ✓ *1/*1 |
| PGXT109 | ✓ *1/*29 | ✓ *1/*1 | ✓ *1/*17 | ✓ *1/*2 | ✓ *1/*5 |
| PGXT110 | ★ *1/*2 + *2 | ✓ *1/*9 | ✓ *1/*1 | ✓ *1/*1 | ✓ *1/*8 |
| PGXT111 | ✓ *4/*35 | ✓ *1/*1 | ✓ *2/*2 | ✓ *1/*4 | ✓ *1/*1 |
| PGXT112 | ✓ *1/*10 + *36 | ✓ *1/*1 | ✓ *1/*2 | ✓ *1/*1 | ✓ *1/*3 |
| PGXT113 | ✓ *1/*2 | \hat{ }✓ *1/*1 | ✓ *2/*17 | ✓ *2/*2 | ✓ *1/*1 |
| PGXT114 | ✓ *1/*2 | ✓ *1/*1 | ✓ *12/*17 | ✓ *2/*2 | ✓ *1/*1 |
| PGXT115 | ✓ *1/*6 | ✓ *1/*1 | ✓ *2/*17 | ✓ *1/*4 | ✓ *1/*1 |
| PGXT116 | ✓ *1/*1 | ✓ *1/*1 | ✓ *2/*2 | ✓ *1/*1 | ✓ *1/*1 |
| PGXT117 | ✓ *2 + *2/*29 | ✓ *17/*20 | ✓ *17/*17 | ✓ *2/*2 | ✓ *1/*1 |
| PGXT118 | ✓ *1/*2 | ✓ *1/*1 | ✓ *1/*1 | ✓ *1/*1 | ✓ *1/*1 |
| PGXT119 | ✓ *4/*40 | \hat{ }✓ *24/*9 | ✓ *1/*2 | ✓ *1/*1 | ✓ *1/*1 |
| PGXT120 | ✓ *1/*1 | ✓ *1/*20 | \hat{ }✓ *27/*6 | ✓ *1/*1 | ✓ *5/*9 |
| PGXT121 | \hat{ }✓ *71/*2 + *2 | \hat{ }✓ *9/? | ✓ *1/*1 | ✓ *1/*1 | ✓ *1/*1 |
| PGXT122 | [symbol] | ✓ *1/*1 | ✓ *1/*1 | ✓ *1/*1 | ✓ *1/*1 |
| PGXT123 | ✓ *2/*17 | ✓ *1/*17 | ✓ *2/*2 | ✓ *1/*1 | ✓ *1/*1 |
| PGXT124 | ✓ *17/*29 | ✓ *1/*17 | ✓ *2/*17 | ✓ *1/*2 | ✓ *1/*1 |
| PGXT125 | ✓ *1/*17 | ✓ *1/*9 | \hat{ }✓ *27/*2 | ✓ *1/*4 | ✓ *1/*9 |
| PGXT126 | ✓ *17/*29 | \hat{ }✓ *23/*9 | ✓ *1/*17 | ✓ *1/*1 | ✓ *1/*1 |
| PGXT127 | ✓ *1/*35 | ✓ *1/*9 | ✓ *1/*1 | ✓ *1/*3 | ✓ *1/*2 |
| PGXT128 | ✓ *1/*4 | \hat{ }✓ *14/*9 | ✓ *2/*17 | ✓ *1/*1 | ✓ *1/*1 |
| PGXT129 | ✓ *10/*10 + *36 | \hat{ }✓ *4/*15 | ✓ *1/*1 | ✓ *1/*1 | ✓ *1/*1 |
| PGXT130 | ✓ *2/*10 + *36 | ✓ *1/*4 | ✓ *1/*1 | ✓ *1/*1 | \hat{ }✓ *1/*18 |
| PGXT131 | ✓ *1/*2 | ✓ *1/*4 | ✓ *1/*1 | ✓ *1/*1 | ✓ *1/*1 |
| PGXT132 | \hat{ }✓ *36 + *36 + | ✓ *1/*1 | ✓ *1/*2 | ✓ *1/*1 | ✓ *1/*1 |
| | *10/*41 | | | | |
| PGXT133 | \hat{ }✓ *1/*21 | ✓ *1/*1 | ✓ *1/*1 | ✓ *1/*1 | ✓ *1/*1 |
| PGXT134 | \hat{ }✓ *2 + *2/*10 | ✓ *1/*17 | ✓ *2/*17 | ✓ *1/*2 | ✓ *1/*1 |
| PGXT135 | \hat{ }✓ *45/*10 | \hat{ }✓ *1/*35 | ✓ *1/*15 | ✓ *1/*1 | ✓ *1/*6 |
| PGXT136 | ✓ *1/*5 | \hat{ }✓ *1/*1 | \hat{ }✓ *27/*2 | ✓ *1/*1 | ✓ *1/*9 |
| PGXT137 | ✓ *1/*6 | ✓ *1/*1 | ✓ *2/*17 | ✓ *1/*4 | ✓ *1/*1 |
| PGXT138 | ✓ *1/*4 | ✓ *1/*1 | ✓ *1/*1 | ✓ *3/*3 | ✓ *2/*2 |
| PGXT139 | ✓ *1/*5 | ✓ *1/*9 | ✓ *1/*17 | ✓ *1/*1 | ✓ *1/*1 |
| PGXT140 | ✓ *1/*5 | \hat{ }✓ *18/*9 | ✓ *1/*1 | ✓ *1/*1 | ✓ *1/*1 |
| PGXT141 | ✓ *2/*10 + *36 | ✓ *9/*9 | ✓ *1/*1 | ✓ *1/*1 | ✓ *1/*1 |
| PGXT142 | ✓ *1/*4 | ✓ *1/*1 | ✓ *1/*17 | ✓ *1/*1 | ✓ *1/*1 |
| PGXT143 | ✓ *1/*9 | ✓ *9/*17 | ✓ *1/*1 | ✓ *1/*1 | ✓ *1/*1 |
| PGXT144 | ✓ *1/*5 | ✓ *1/*1 | ✓ *1/*1 | ✓ *1/*1 | ✓ *1/*1 |
| PGXT145 | ✓ *4/*4 + *4 | ✓ *1/*1 | ✓ *1/*2 | ✓ *1/*4 | ✓ *10/*12 |
| PGXT146 | ✓ *4/*5 | ✓ *1/*2 | ✓ *1/*17 | ✓ *1/*1 | ✓ *1/*2 |
| PGXT147 | ✓ *15/*17 | ✓ *1/*17 | ✓ *13/*17 | ✓ *1/*2 | ✓ *1/*1 |
| PGXT148 | ✓ *4/*35 | \hat{ }✓ *1/*1 | ✓ *1/*1 | ✓ *1/*3 | ✓ *1/*2 |
| PGXT149 | ✓ *2/*10 + *36 | ✓ *1/*1 | ✓ *2/*3 | ✓ *1/*1 | ✓ *1/*1 |
| PGXT150 | ✓ *1/*4 | ✓ *1/*1 | ✓ *1/*1 | ✓ *1/*1 | ✓ *1/*2 |
| PGXT151 | ✓ *1/*35 | ✓ *1/*1 | ✓ *8/*17 | ✓ *1/*3 | ✓ *1/*2 |
| PGXT152 | ✓ *1/*4 | ✓ *1/*2 | ✓ *1/*17 | ✓ *1/*1 | ✓ *1/*1 |
| PGXT153 | ✓ *29/*29 | ✓ *1/*1 | ✓ *2/*9 | ✓ *1/*1 | ✓ *1/*1 |
| PGXT154 | ✓ *1/*40 | ✓ *1/*1 | ✓ *1/*1 | ✓ *1/*1 | ✓ *5/*5 |
| PGXT155 | \hat{ }✓ *1/*68 + *4 | [symbol] *1/*12? | ✓ *1/*8 | ✓ *3/*3 | ✓ *2/*2 |
| PGXT156 | \hat{ }✓ *2/*41 + | ✓ *1/*1 | ✓ *1/*1 | ✓ *1/*3 | ✓ *1/*2 |
| | *41 + *41 | | | | |
| PGXT157 | ✓ *15 + *41 | ✓ *1/*1 | ✓ *1/*2 | ✓ *1/*4 | ✓ *1/*1 |
| PGXT158 | ✓ *1/*68 + *4 | ✓ *1/*9 | ✓ *9/*17 | ✓ *1/*1 | ✓ *1/*1 |
| PGXT159 | ✓ *7/*35 | ✓ *1/*1 | ✓ *8/*17 | ✓ *1/*3 | ✓ *1/*2 |
| PGXT160 | \hat{ }✓ *4/*68 + *4 | ✓ *1/*1 | ✓ *2/*6 | ✓ *1/*1 | ✓ *1/*1 |
| PGXT161 | \hat{ }✓ *10 + *36/ | ✓ *1/*1 | ✓ *3/*17 | ✓ *1/*1 | ✓ *1/*1 |
| | *10 + *10 | | | | |
| PGXT162 | ✓ *2/*2 + *2 | ✓ *1/*1 | ✓ *1/*17 | ✓ *1/*1 | ✓ *1/*1 |
| PGXT163 | \hat{ }✓ *3/*68 + *4 | ✓ *1/*1 | ✓ *1/*2 | ✓ *1/*3 | ✓ *1/*2 |
| PGXT164 | ✓ *2/*4 | ✓ *1/*1 | ✓ *1/*17 | ✓ *1/*1 | \hat{ }✓ *1/*18 |
| PGXT165 | \hat{ }✓ *1/*21 | ✓ *4/*4 | ✓ *1/*1 | ✓ *1/*1 | ✓ *1/*1 |
| PGXT166 | ✓ *4/*5 | ✓ *1/*1 | ✓ *1/*1 | ✓ *1/*3 | ✓ *1/*2 |
| PGXT167 | \hat{ }✓ *68 + *4/*5 | ✓ *1/*1 | ✓ *1/*2 | ✓ *1/*3 | ✓ *1/*2 |
| PGXT168 | \hat{ }✓ *4 + *4/*41 | ✓ *1/*1 | \hat{ }✓ *2/*10$ | ✓ *1/*1 | ✓ *1/*9 |
| PGXT169 | ✓ *1/*9 | \hat{ }✓ *14/*9 | ✓ *1/*1 | ✓ *1/*1 | ✓ *1/*2 |
| PGXT170 | ✓ *1/*4 | ✓ *1/*1 | ✓ *1/*1 | ✓ *1/*1 | ✓ *1/*1 |
| PGXT171 | ✓ *2/*2 | ✓ *1/*4 | ✓ *1/*1 | ✓ *1/*1 | ✓ *1/*1 |
| PGXT172 | ✓ *1/*1 | ✓ *1/*1 | ✓ *1/*1 | ✓ *1/*3 | ✓ *1/*2 |
| PGXT173 | ✓ *1/*41 | ✓ *1/*1 | ✓ *1/*1 | ✓ *1/*1 | ✓ *1/*1 |
| PGXT174 | ✓ *4/*41 | ✓ *1/*1 | ✓ *1/*1 | ✓ *1/*1 | ✓ *1/*1 |
| PGXT175 | ✓ *4/*4 | ✓ *1/*1 | ✓ *1/*17 | ✓ *1/*1 | ✓ *1/*1 |
| PGXT176 | \hat{ }✓ *1/*10 + | ✓ *1/*9 | ✓ *1/*1 | ✓ *1/*1 | ✓ *1/*1 |
| | *36 + *36 | | | | |
| PGXT177 | ✓ *2/*4 | ✓ *1/*1 | ✓ *1/*1 | ✓ *1/*1 | ✓ *1/*1 |
| PGXT178 | ✓ *1/*4 | ✓ *1/*1 | ✓ *2/*17 | ✓ *1/*4 | ✓ *1/*1 |
| PGXT179 | ✓ *1/*35 | ✓ *1/*1 | ✓ *1/*1 | ✓ *1/*1 | \hat{ }✓ *1/*18 |
| PGXT180 | \hat{ }✓*1/*28 | ✓ *1/*1 | ✓ *1/*13 | ✓ *1/*2 | ✓ *1/*1 |
| PGXT181 | ✓ *1/*41 | ✓ *1/*9 | ✓ *1/*1 | ✓ *1/*1 | \hat{ }✓ *1/*18 |
| PGXT182 | ✓ *2/*5 | ✓ *1/*17 | ✓ *1/*2 | ✓ *1/*1 | ✓ *1/*1 |
| PGXT183 | ✓ *1/*29 | ✓ *9/*9 | ✓ *1/*1 | ✓ *1/*2 | ✓ *1/*1 |
| PGXT184 | ✓ *2/*17 | \hat{ }✓ *1/*35 | \hat{ }✓ *15/*2 | ✓ *1/*1 | ✓ *1/*11 |
| PGXT185 | ✓ *1/*1 | ✓ *1/*1 | ✓ *1/*2 | ✓ *1/*1 | ✓ *1/*1 |
| PGXT186 | ✓ *2/*2 + *2 | ✓ *1/*17 | ✓ *1/*2 | ✓ *1/*1 | ✓ *1/*8 |
| PGXT187 | ✓ *10/*41 | [symbol] *7/*18 | ✓ *1/*2 | ✓ *1/*1 | ✓ *1/*1 |
| PGXT188 | ✓ *2/*2 | ✓ *4/*4 | ✓ *1/*1 | ✓ *1/*1 | ✓ *1/*1 |
| PGXT189 | \hat{ }✓ *1/*68 + *4 | ✓ *1/*2 | ✓ *1/*2 | ✓ *1/*1 | \hat{ }✓ *1/*18 |
| PGXT190 | ✓ *5/*29 | [symbol] *1/*12? | ✓ *1/*1 | ✓ *1/*1 | ✓ *1/*1 |
| PGXT191 | %✓ *1/*46 | ✓ *1/*17 | ✓ *1/*17 | ✓ *1/*1 | ✓ *1/*5 |
| PGXT192 | ✓ *2/*4 + *4 | ✓ *1/*1 | ✓ *1/*17 | ✓ *1/*2 | ✓ *1/*1 |
| PGXT193 | ✓ *1/*4 + *4 | \hat{ }✓ *1/*35 | ✓ *1/*1 | ✓ *1/*1 | ✓ *1/*1 |
| PGXT194 | ✓ *1/*1 | ✓ *1/*4 | ✓ *1/*1 | ✓ *1/*1 | ✓ *1/*1 |
| PGXT195 | ✓ *1/*1 | \hat{ }✓ *24/*17 | ✓ *1/*15 | ✓ *1/*1 | ✓ *1/*6 |
| PGXT196 | ✓ *1/*14 | ✓ *9/*9 | ✓ *1/*4 | ✓ *1/*1 | ✓ *1/*1 |
| PGXT201 | ✓ *1/*41 | ✓ *1/*9 | ✓ *8/*17 | ✓ *1/*3 | ✓ *1/*2 |
| PGXT202 | ✓ *4/*29 | \hat{ }✓ *35/*17 | \hat{ }✓ *1/*27 | ✓ *1/*1 | ✓ *1/*9 |
| PGXT203 | ✓ *1/*1 | ✓ *1/*1 | ✓ *2/*17 | ✓ *1/*4 | ✓ *1/*1 |
| PGXT204 | ✓ *1/*41 | ✓ *1/*1 | ✓ *1/*2 | ✓ *1/*1 | \hat{ }✓ *1/*18 |
| PGXT205 | ✓ *5/*41 | [symbol] 1/*12? | ✓ *17/*17 | ✓ *1/*1 | ✓ *1/*1 |
| PGXT206 | \hat{ }✓ *1/*36 + *10 | ✓ *1/*1 | ✓ *2/*2 | ✓ *1/*1 | ✓ *1/*1 |
| PGXT207 | ✓ *1/*40 | ✓ *1/*1 | ✓ *1/*17 | ✓ *1/*1 | \hat{ }✓ *36/*5 |
| PGXT208 | ✓ *1/*1 | ✓ *17/*17 | ✓ *1/*17 | ✓ *1/*2 | ✓ *1/*1 |
| PGXT209 | *1/*4 | ★ *1/*21 | ✓ *1/*17 | ✓ *1/*1 | ✓ *1/*1 |
| PGXT210 | \hat{ }✓ *10 + *36/*10 + | ✓ *1/*4 | ✓ *1/*2 | ✓ *1/*1 | ✓ *1/*1 |
| | *36 | | | | |
| PGXT211 | \hat{ }✓ *1/*10 + *36 | ✓ *1/*9 | ✓ *2/*2 | ✓ *1/*1 | ✓ *1/*1 |
| PGXT212 | \hat{ }✓ *5/*10$ | ✓ *1/*1 | ✓ *1/*2 | ✓ *1/*1 | ✓ *1/*1 |
| PGXT213 | ✓ *2/*3 | ✓ *1/*1 | \hat{ }✓ *27/*2 | ✓ *1/*4 | ✓ *1/*9 |
| PGXT214 | \hat{ }✓ *35/*4 | ✓ *1/*1 | ✓ *1/*1 | ✓ *1/*3 | ✓ *1/*2 |
| PGXT215 | \hat{ }✓ *1/*68 + *4 | ✓ *1/*2 | ✓ *1/*1 | ✓ *1/*3 | \hat{ }✓ *1/*18 |
| PGXT216 | ✓ *2/*41 | [symbol] *1/*12? | ✓ *1/*2 | ✓ *1/*1 | ✓ *1/*8 |
| PGXT217 | \hat{ }✓ *1/*10 + *36 | ✓ χ*1/*1 | ✓ *1/*1 | ✓ *1/*1 | \hat{ }✓ *1/*18 |
| PGXT218 | \hat{ }✓*10 + *36/*41 | ✓ *1/*1 | ✓ *1/*1 | ✓ *1/*1 | ✓ *1/*1 |
| PGXT219 | \hat{ }✓ *9/*35 | ✓ *1/*1 | ✓ *1/*17 | ✓ *1/*1 | ✓ *1/*1 |
| PGXT220 | ✓ *2/*3 | ✓ *1/*1 | ✓ *1/*17 | ✓ *1/*1 | ✓ *1/*1 |
| PGXT221 | ✓ *5/*17 | ✓ *1/*9 | ✓ *1/*2 | ✓ *1/*1 | ✓ *1/*8 |
| PGXT222 | ✓ *2/*5 | ✓ *9/*17 | ✓ *17/*17 | ✓ *2/*2 | ✓ *1/*1 |
| PGXT223 | ✓ *2/*4 | ✓ *1/*1 | ✓ *1/*1 | ✓ *1/*1 | ✓ *1/*1 |
| PGXT224 | ✓ *1/*5 | ✓ *1/*1 | ✓ *1/*2 | ✓ *1/*1 | ✓ *1/*2 |
| PGXT225 | ✓ *1/*17 | ✓ *1/*9 | ✓ *1/*1 | ✓ *1/*1 | ✓ *1/*1 |
| PGXT226 | ✓ *2 + *2/*6 | ★ *1/*35 | ✓ *1/*2 | ✓ *1/*3 | ✓ *1/*2 |
| PGXT227 | ✓ *83/*4 + *4 | ✓ *1/*1 | \hat{ }✓ *4/*17 | ✓ *1/*1 | ✓ *1/*1 |
| PGXT228 | \hat{ }✓ *2 + *2/*4 | \hat{ }✓ *1/*14 | ✓ *1/*1 | ✓ *1/*1 | ✓ *1/*1 |
| PGXT229 | ✓ *10 + *10/*17 | ✓ *1/*1 | ✓ *1/*17 | ✓ *1/*2 | ✓ *1/*1 |
| PGXT230 | ✓ *1/*7 | ✓ *1/*1 | ✓ *1/*2 | ✓ *1/*1 | \hat{ }✓ *1/*18 |
| PGXT231 | ✓ *2/*41 | ✓ *1/*1 | ✓ *1/*4 | ✓ *1/*3 | ✓ *1/*2 |
| PGXT232 | ✓ *2/*2 | ✓ *1/*1 | ✓ *1/*8 | ✓ *1/*3 | ✓ *1/*2 |
| PGXT233 | ✓ *2/*3 | ✓ *1/*1 | ✓ *1/*1 | ✓ *1/*3 | ✓ *1/*2 |
| PGXT234 | ✓ *1/*5 | \hat{ }✓ *4/*9 | ✓ *1/*2 | ✓ *1/*1 | ✓ *1/*1 |
| PGXT235 | ✓ *1/*2 | ✓ *1/*2 | ✓ *1/*1 | ✓ *1/*1 | ✓ *1/*1 |
| PGXT236 | ✓ *1/*1 | ✓ *1/*1 | ✓ *2/*17 | ✓ *1/*1 | ✓ *1/*1 |
| PGXT237 | ✓ *1/*4 | ✓ *1/*1 | ✓ *1/*17 | ✓ *1/*1 | ✓ *1/*1 |
| PGXT238 | \hat{ }✓ *1/*79 + *2 | ✓ *1/*1 | ✓ *1/*1 | ✓ *1/*1 | ✓ *1/*1 |
| PGXT239 | ✓ *1/*40 | ✓ *1/*1 | \hat{ }✓ *15/*2 | ✓ *1/*1 | \hat{ }✓ *1/*18 |
| PGXT240 | \hat{ }✓ *2 + *2/*68 + *4 | \hat{ }✓ *1/*21 | ✓ *1/*2 | ✓ *1/*1 | ✓ *1/*1 |
| PGXT241 | ✓ *1/*2 | ✓ *17/*17 | ✓ *2/*2 | ✓ *1/*1 | ✓ *1/*1 |

| Sample | CYP3A4 | CYP3A5 | CYP4F2 | TPMT | DPYD |
|---|---|---|---|---|---|
| Concordance | 131 | 137 | 126 | 135 | 79 |
| Improvement | 6 | 0 | 11 0 | 2 | 53 |
| Unknown | 0 | 0 | 0 | 0 | 2 |
| Failure | | | | | |
| PGXT101 | ✓ *1/*1 | ✓ *3/*3 | ✓ *1/*1 | ✓ *1/*1 | ✓ *9/*9 |
| PGXT102 | ✓ *1/*1B | ✓ *1/*3 | ✓ *1/*3 | ✓ *1/*1 | \hat{ }✓ *1/*5 |
| PGXT103 | ✓ *1/*1 | ✓ *3/*3 | ✓ *1/*1 | ✓ *1/*3A | \hat{ }✓ *1/*5 |
| PGXT104 | ✓ *1/*1 | ✓ *3/*3 | ✓ *1/*1 | ✓ *1/*1 | \hat{ }✓ *5/*9 |
| PGXT105 | ✓ *1/*1 | ✓ *1/*3 | %✓ *2/*3 | ✓ *1/*1 | \hat{ }✓ *1/*5 |
| PGXT106 | ✓ *1/*1B | ✓ *3/*3 | %✓ *2/*3 | ✓ χ*1/*1 | ✓ *1/*9 |
| PGXT107 | ✓ *1/*1 | ✓ *3/*3 | ✓ *1/*1 | ✓ *1/*1 | ★ *5/*6 |
| PGXT108 | ✓ *1/*1 | ✓ *3/*3 | ✓ *1/*1 | ✓ *1/*3A | \hat{ }✓ *4/*6 |
| PGXT109 | ✓ *1B/*1B | ✓ *1/*6 | %✓ *2/*3 | ✓ *1/*1 | ✓ *9/*9 |
| PGXT110 | ✓ *1/*1 | ✓ *1/*3 | ✓ *1/*3 | ✓ *1/*1 | \hat{ }✓ *1/*5 |
| PGXT111 | ✓ *1/*1 | ✓ *3/*3 | ✓ *1/*1 | ✓ *1/*1 | ✓ *1/*9 |
| PGXT112 | ✓ *1/*1 | ✓ *1/*3 | ✓ *1/*3 | ✓ χ*1/*1 | \hat{ }✓ *1/*5 |
| PGXT113 | ✓ *1B/*1B | ✓ *1/*3 | ✓ *1/*1 | ✓ *1/*8 | ✓ *1/*1 |
| PGXT114 | ✓ *1/*1 | ✓ *3/*3 | %✓ *2/*3 | ✓ *1/*1 | ✓ *1/*5 |
| PGXT115 | ✓ *1/*1 | ✓ *3/*3 | ✓ *1/*1 | ✓ *1/*1 | [symbol] |
| PGXT116 | ✓ *1B/*22 | ✓ *1/*3 | ✓ *1/*3 | ✓ *1/*1 | ✓ *1/*1 |
| PGXT117 | \hat{ }✓ *1B/*15 | ✓ *1/*3 | ✓ *1/*1 | ✓ *1/*1 | ✓ *1/*9 |
| PGXT118 | ✓ *1B/*1B | ✓ *1/*6 | ✓ *1/*1 | ✓ *1/*3C | ✓ *1/*9 |
| PGXT119 | ✓ *1B/*1B | ✓ *1/*6 | ✓ *1/*1 | ✓ *1/*1 | ✓ *1/*1 |
| PGXT120 | ✓ *1B/*1B | ✓ *1/*1 | ✓ *1/*1 | ✓ *1/*1 | ✓ *1/*9 |
| PGXT121 | ✓ *1/*1 | ✓ *3/*3 | %✓ *2/*3 | ✓ *1/*1 | ✓ *1/*1 |
| PGXT122 | ✓ *1/*1 | ✓ *3/*3 | ✓ *1/*1 | ✓ *1/*1 | ✓ *1/*9 |
| PGXT123 | ✓ *1/*1B | ✓ *1/*7 | %✓ *2/*3 | ✓ *1/*1 | ★ *5/*9 |
| PGXT124 | ✓ *1B/*1B | ✓ *1/*6 | ✓ *1/*2 | ✓ *1/*1 | \hat{ }✓ *5/*9 |
| PGXT125 | ✓ *1B/*1B | ✓ *1/*7 | ✓ *1/*2 | ✓ *1/*1 | ✓ *1/*9 |
| PGXT126 | ✓ *1/*1B | ✓ *1/*3 | ✓ *1/*1 | ✓ *1/*1 | ✓ *1/*9 |
| PGXT127 | ✓ *1/*1 | ✓ *3/*3 | ✓ *1/*1 | ✓ *1/*3A | ✓ *1/*9 |
| PGXT128 | ✓ *1/*1 | ✓ *3/*3 | %✓ *2/*3 | ✓ *1/*1 | ✓ *1/*9 |
| PGXT129 | ✓ *1/*1 | ✓ *1/*3 | ✓ *1/*1 | ✓ *1/*1 | ✓ *1/*1 |
| PGXT130 | ✓ *1/*1 | ✓ *1/*3 | ✓ *1/*1 | ✓ *1/*1 | ✓ *1/*1 |
| PGXT131 | \hat{ }✓ *1/*16 | ✓ *1/*3 | ✓ *1/*3 | ✓ *1/*3C | ✓ *1/*1 |
| PGXT132 | ✓ *1/*1 | ✓ *1/*3 | ✓ *1/*3 | ✓ *1/*1 | ✓ *1/*9 |
| PGXT133 | ✓ *1/*1 | ✓ *3/*3 | ✓ *1/*1 | ✓ *1/*3C | \hat{ }✓ *1/*5 |
| PGXT134 | ✓ *1B/*1B | ✓ *3/*7 | ✓ *1/*1 | ✓ *1/*1 | ✓ *1/*9 |
| PGXT135 | ✓ *1B/*1B | ✓ *6/*7 | ✓ *1/*1 | ✓ *1/*1 | ✓ *1/*1 |
| PGXT136 | ✓ *1/*1B | ✓ *3/*6 | ✓ *1/*1 | ✓ *1/*3C | ✓ *9/*9 |
| PGXT137 | ✓ *1/*1 | ✓ *3/*3 | ✓ *1/*1 | ✓ *1/*1 | \hat{ }✓ *6/*9 |
| PGXT138 | ✓ *1/*1B | ✓ *1/*3 | ✓ *1/*3 | ✓ *1/*1 | ✓ *1/*1 |
| PGXT139 | ✓ *1/*1 | ✓ *3/*3 | ✓ *1/*1 | ✓ *1/*1 | ✓ *1/*1 |
| PGXT140 | ✓ *1/*1 | ✓ *3/*3 | ✓ *1/*1 | ✓ *1/*1 | ✓ *1/*1 |
| PGXT141 | ✓ *1/*1 | ✓ *1/*3 | ✓ *1/*1 | ✓ *1/*1 | \hat{ }✓ *1/*5 |
| PGXT142 | ✓ *1/*1 | ✓ *3/*3 | ✓ *1/*3 | ✓ *1/*1 | ✓ *1/*1 |
| PGXT143 | ✓ *1/*1 | ✓ *3/*3 | ✓ *1/*3 | ✓ *1/*1 | \hat{ }✓ *1/*5 |
| PGXT144 | ✓ *1/*1 | ✓ *3/*3 | ✓ *1/*1 | ✓ *1/*1 | \hat{ }✓ *1/*5 |
| PGXT145 | \hat{ }✓ *1/*14 | ✓ *3/*3 | ✓ *1/*1 | ✓ *1/*3A | \hat{ }✓ *1/*5 |
| PGXT146 | ✓ *1/*1 | ✓ *3/*3 | ✓ *1/*1 | ✓ *1/*1 | \hat{ }✓ *5/*9 |
| PGXT147 | ✓ *1/*1B | ✓ *1/*1 | ✓ *1/*1 | ✓ *1/*1 | ✓ *1/*9 |
| PGXT148 | ✓ *1/*1B | ✓ *1/*3 | ✓ *1/*1 | ✓ *1/*1 | ✓ *1/*1 |
| PGXT149 | ✓ *1/*1 | ✓ *1/*1 | \hat{ }✓ *3/*4 | ✓ *1/*1 | \hat{ }✓ *5/*9 |
| PGXT150 | ✓ *1/*1 | ✓ *3/*3 | %✓ *2/*3 | ✓ *1/*1 | \hat{ }✓ *1/*5 |
| PGXT151 | ✓ *1/*1 | ✓ *1/*3 | ✓ *3/*4 | ✓ *1/*1 | ✓ *1/*1 |
| PGXT152 | ✓ *1/*1 | ✓ *3/*3 | ✓ *1/*1 | ✓ *1/*1 | ✓ *1/*9 |
| PGXT153 | ✓ *1/*1B | *3/*6 | ✓ *2/*3 | ✓ *1/*8 | \hat{ }✓ *1/*5 |
| PGXT154 | ✓ *1B/*1B | ✓ *1/*1 | ✓ *1/*1 | ✓ *1/*8 | \hat{ }✓ *1/*5 |
| PGXT155 | ✓ *1/*1 | ✓ *3/*3 | ✓ *2/*3 | ✓ *1/*1 | \hat{ }✓ *6/*2 |
| PGXT156 | ✓ *1/*1 | ✓ *1/*3 | ✓ *3/*3 | ✓ *1/*1 | ✓ *4/*9 |
| PGXT157 | ✓ *1/*1 | ✓ *3/*3 | ✓ *1/*1 | ✓ *1/*1 | ✓ *1/*1 |
| PGXT158 | ✓ *22/*22 | ✓ *3/*3 | %✓ *2/*3 | ✓ *1/*1 | \hat{ }✓ *1/*5 |
| PGXT159 | ✓ *1/*1 | ✓ *3/*3 | %✓ *2/*3 | ✓ *1/*1 | \hat{ }✓ *1/*5 |
| PGXT160 | ✓ *1/*1 | ✓ *3/*3 | ✓ *1/*1 | ✓ *1/*1 | \hat{ }✓ *1/*5 |
| PGXT161 | ✓ *1/*1 | ✓ *3/*3 | ✓ *1/*3 | ✓ *1/*1 | \hat{ }✓ *1/*5 |
| PGXT162 | ✓ *1/*22 | ✓ *3/*3 | \hat{ }✓ *1/*1 | ✓ *1/*1 | ✓ *1/*1 |
| PGXT163 | ✓ *1/*1 | ✓ *3/*3 | ✓ *1/*1 | ✓ *1/*1 | \hat{ }✓ *4/*5 |
| PGXT164 | ✓ *1/*1 | ✓ *3/*3 | ✓ *1/*3 | ✓ *1/*1 | ✓ *1/*4 |
| PGXT165 | ✓ *1/*1 | ✓ *1/*3 | ✓ *1/*1 | ✓ *1/*1 | \hat{ }✓ *5/*9 |
| PGXT166 | ✓ *1/*2 | ✓ *3/*3 | ✓ *1/*1 | \hat{ }✓ *1S/*16 | ✓ *1/*1 |
| PGXT167 | ✓ *1/*1B | ✓ *1/*1 | ✓ *1/*3 | ✓ *1/*1 | ✓ *1/*9 |
| PGXT168 | ✓ *1/*1 | ✓ *1/*1 | ✓ *1/*2 | ✓ *1/*1 | ✓ *1/*1 |
| PGXT169 | ✓ *1/*1 | ✓ *3/*3 | %✓ *2/*3 | ✓ *1/*1 | ✓ *1/*1 |
| PGXT170 | ✓ *1/*1 | ✓ *3/*3 | ✓ *1/*1 | ✓ *1/*1 | ✓ *1/*4 |
| PGXT171 | ✓ *1/*1 | ✓ *3/*3 | ✓ *1/*1 | ✓ *1/*1 | ✓ *1/*1 |
| PGXT172 | ✓ *1/*1 | ✓ *3/*3 | %✓ *2/*3 | ✓ *1/*1 | \hat{ }✓ *1/*5 |
| PGXT173 | ✓ *1/*1 | ✓ χ*3/*3 | ✓ *1/*1 | ✓ *1/*1 | \hat{ }✓ *5/*9 |
| PGXT174 | ✓ *1/*3 | ✓ *3/*3 | ✓ *1/*3 | ✓ *1/*1 | ✓ *1/*1 |
| PGXT175 | ✓ *1/*1 | ✓ *3/*3 | ✓ *1/*1 | ✓ *1/*1 | \hat{ }✓ *1/*5 |
| PGXT176 | ✓ *1/*1 | ✓ *1/*1 | ✓ *1/*3 | ✓ *1/*1 | ✓ *1/*1 |
| PGXT177 | ✓ *1/*22 | ✓ *3/*3 | ✓ *3/*4 | ✓ *1/*1 | ✓ *1/*1 |
| PGXT178 | ✓ *1/*1 | ✓ *3/*3 | ✓ *1/*1 | ✓ *1/*1 | ✓ *1/*9 |
| PGXT179 | ✓ *1/*1 | ✓ *3/*3 | ✓ *1/*1 | ✓ *1/*1 | \hat{ }✓ *5/*9 |
| PGXT180 | ✓ *1/*1 | ✓ *3/*3 | ✓ *3/*3 | ✓ *1/*1 | \hat{ }✓ *5/*9 |
| PGXT181 | ✓ *1/*1 | ✓ *3/*3 | ✓ *1/*1 | ✓ *1/*1 | \hat{ }✓ *1/*6 |
| PGXT182 | ✓ *1B/*1B | ✓ *1/*3 | ✓ *1/*1 | ✓ *1/*1 | ✓ *1/*9 |
| PGXT183 | ✓ *1/*1B | ✓ *1/*3 | ✓ *1/*3 | ✓ *1/*1 | ✓ *9/*9 |
| PGXT184 | ✓ *1B/*1B | ✓ *1/*1 | ✓ *1/*2 | ✓ *1/*1 | ✓ *1/*1 |
| PGXT185 | ✓ *1/*1 | ✓ *3/*3 | ✓ *1/*1 | ✓ *1/*1 | ✓ *1/*1 |
| PGXT186 | \hat{ }✓ *1B/*15 | ✓ *1/*6 | ✓ *1/*2 | ✓ *1/*1 | ✓ *1/*9 |
| PGXT187 | ✓ *1/*1 | ✓ *1/*3 | ✓ *1/*1 | ✓ *1/*1 | \hat{ }✓ *1/*5 |
| PGXT188 | ✓ *1/*1 | ✓ *3/*3 | ✓ *1/*1 | ✓ *1/*1 | \hat{ }✓ *5/*5 |
| PGXT189 | ✓ *1/*1 | ✓ *3/*3 | ✓ *1/*1 | ✓ *1/*1 | \hat{ }✓ *1/*5 |
| PGXT190 | ✓ *1B/*1B | ✓ *1/*1 | ✓ *1/*1 | ✓ *1/*1 | ✓ *1/*9 |
| PGXT191 | \hat{ }✓ *1B/*15 | ✓ *1/*3 | %✓ *2/*3 | ✓ *1/*1 | ✓ *1/*9 |
| PGXT192 | ✓ *1/*1B | ✓*3/*6 | %✓ *2/*3 | ✓ *1/*1 | \hat{ }✓ *5/*9 |
| PGXT193 | ✓ *1B/*1B | ✓ *7/*7 | ✓ *1/*1 | ✓ *1/*3C | ✓ *9/*9 |
| PGXT194 | ✓ *1/*1 | ✓ *3/*3 | ✓ *1/*3 | ✓ *1/*1 | ✓ *1/*1 |
| PGXT195 | ✓ *1B/*1B | ✓ *1/*6 | ✓ *1/*1 | ✓ *1/*1 | ✓ *1/*1 |
| PGXT196 | ✓ *1/*1 | ✓ *3/*3 | ✓ *1/*3 | ✓ *1/*1 | \hat{ }✓ *1/*5 |
| PGXT201 | ✓ *1/*1 | ✓ *3/*3 | ✓ *1/*1 | ✓ *1/*1 | ✓ *1/*1 |
| PGXT202 | ✓ *1/*1 | ✓ *1/*3 | \hat{ }✓ *1/*2 | ✓ *1/*1 | ✓ *9/*9 |
| PGXT203 | ✓ *1/*1 | ✓ *3/*3 | ✓ *1/*3 | ✓ *1/*1 | \hat{ }✓ *5/*9 |
| PGXT204 | ✓ *1/*1 | ✓ *3/*3 | ✓ *3/*3 | ✓ *1/*1 | \hat{ }✓ *1/*5 |
| PGXT205 | ✓ *1/*1 | ✓ *3/*3 | ✓ *1/*1 | ✓ *1/*1 | ✓ *1/*9 |
| PGXT206 | ✓ *1/*1 | ✓ *1/*3 | ✓ *3/*3 | ✓ *1/*1 | ✓ *1/*1 |
| PGXT207 | ✓ *1/*1 | ✓ *3/*3 | %✓ *2/*3 | ✓ *1/*1 | \hat{ }✓ *5/*9 |
| PGXT208 | ✓ *1/*1 | ✓ *3/*7 | \hat{ }✓ *1/*2 | ✓ *1/*1 | \hat{ }✓ *1/*5 |
| PGXT209 | ✓ *1/*1 | ✓ *3/*3 | ✓ *1/*1 | ✓ *1/*1 | ✓ *1/*1 |
| PGXT210 | ✓ *1/*1 | ✓ *3/*3 | ✓ *1/*1 | ✓ *1/*1 | ✓ *1/*1 |
| PGXT211 | ✓ *1/*1 | ✓ *1/*3 | ✓ *1/*1 | ✓ *1/*1 | ✓ *1/*1 |
| PGXT212 | ✓ *1/*1 | ✓ *3/*3 | ✓ *1/*1 | ✓ *1/*1 | \hat{ }✓ *1/*5 |
| PGXT213 | ✓ *1/*1 | ✓ *3/*3 | ✓ *1/*1 | ✓ *1/*3A | ✓ *1/*9 |
| PGXT214 | ✓ *1/*22 | ✓ *3/*3 | ✓ *1/*1 | ✓ *1/*1 | \hat{ }✓ *4/*5 |
| PGXT215 | ✓ *1/*1 | ✓ *3/*3 | ✓ *1/*1 | ✓ *1S/*32 | \hat{ }✓ *1/*5 |
| PGXT216 | ✓ *1/*1 | ✓ *3/*3 | ✓ *1/*3 | ✓ *1/*1 | ✓ *1/*9 |
| PGXT217 | ✓ *1/*1 | ✓ *1/*1 | ✓ *1/*1 | ✓ *1/*1 | \hat{ }✓ *1/*5 |
| PGXT218 | ✓ *1/*1 | ✓ *1/*3 | ✓ *1/*3 | ✓ *1/*1 | \hat{ }✓ *1/*5 |
| PGXT219 | ✓ *1/*1 | ✓ *1/*3 | ✓ *3/*4 | ✓ *1/*1 | ✓ *1/*1 |
| PGXT220 | ✓ *1/*1 | ✓ *3/*3 | %✓ *2/*3 | ✓ *1/*1 | \hat{ }✓ *1/*5 |
| PGXT221 | ✓ *1/*1 | ✓ *1/*1 | ✓ *1/*1 | ✓ *1/*1 | \hat{ }✓ *5/*9 |
| PGXT222 | \hat{ }✓ *1/*12 | ✓ *1/*7 | ✓ *1/*3 | ✓ *3C/*3C | ✓ *1/*1 |
| PGXT223 | ✓ *1/*1 | ✓ *3/*3 | ✓ *1/*1 | ✓ *1/*1 | ✓ *1/*1 |
| PGXT224 | ✓ *1/*1 | ✓ *1/*3 | ✓ *1/*1 | ✓ *1/*1 | \hat{ }✓ *5/*6 |
| PGXT225 | ✓ *1/*1 | ✓ *1/*1 | ✓ *1/*2 | ✓ *1/*1 | ✓ *9/*9 |
| PGXT226 | ✓ *1/*1 | ✓ *1/*3 | ✓ *1/*1 | ✓ *1/*1 | [symbol] |
| PGXT227 | ✓ *1/*1 | ✓ *3/*3 | ✓ *1/*3 | ✓ *1/*1 | ✓ *1/*1 |
| PGXT228 | ✓ *1/*1 | ✓ *3/*3 | ✓ *1/*3 | ✓ *1/*1 | \hat{ }✓ *5/*5 |
| PGXT229 | ✓ *1/*1 | ✓ *1/*7 | ✓ *1/*1 | ✓ *1/*1 | ✓ *1/*9 |
| PGXT230 | ✓ *1/*1 | ✓ *1/*3 | ✓ *1/*3 | ✓ *1/*1 | ✓ *1/*1 |
| PGXT231 | ✓ *1/*1 | ✓ *3/*3 | ✓ *1/*1 | ✓ *1/*1 | ✓ *1/*1 |
| PGXT232 | ✓ *1/*1 | ✓ *3/*3 | %✓ *2/*3 | ✓ *1/*1 | ✓ *9/*9 |
| PGXT233 | ✓*1/*22 | ✓ *3/*3 | ✓ *1/*1 | ✓ *1/*1 | \hat{ }✓ *1/*5 |
| PGXT234 | ✓ *1/*1 | ✓ *1/*3 | ✓ *1/*1 | ✓ *1/*1 | ✓ *1/*1 |
| PGXT235 | ✓ *1/*1 | ✓ *3/*3 | %✓ *2/*3 | ✓ *1/*1 | [symbol] |
| PGXT236 | ✓ *1/*1 | ✓ *3/*3 | ✓ *1/*1 | ✓ *1/*1 | ✓ *1/*1 |
| PGXT237 | ✓ *1/*1 | ✓ *3/*3 | ✓ *1/*3 | ✓ *1/*1 | ✓ *1/*9 |
| PGXT238 | ✓ *1/*1 | ✓ *1/*3 | ✓ *3/*3 | ✓ *1/*1 | ✓ *1/*1 |
| PGXT239 | ✓ *1/*1 | ✓ *1/*7 | \hat{ }✓ *1/*2 | ✓ *1/*1 | ✓ *9/*9 |
| PGXT240 | ✓ *1/*1 | ✓ *3/*3 | ✓ *1/*1 | ✓ *1/*1 | \hat{ }✓ *1/*5 |
| PGXT241 | ✓ *1/*1 | ✓ *3/*3 | %✓ *2/*3 | ✓ *1/*3C | ✓ *1/*9 |

M = mother; F = father; C = child.
Concordance is indicated by ✓: matches panel validation.
Improvement is indicated by \hat{ }✓: improvements over panel validations.
Unknown is indicated by ★: unsure of proper genotype.
Failure is indicated by [symbol] : present methods did not identify optimal genotype.
Samples where the present methods identified multiple optimal solutions are indicated by %✓.

### Present Methods Outperform Other Available Methods

The present methods correctly called CYP2D6 genotypes for all samples, significantly outperforming Cypiripi, which was unable to properly genotype about 50% of the samples, and Astrolabe (formerly Constellation), which misidentified about 40% of the samples (see Table 6). Both Astrolabe and Cypiripi currently only support the CYP2D6 gene, and thus this gene was used as a means for comparison. The results in the top half of Table 6 depict the superior performance of the present methods on the 96 PGRNseq v.2 samples from data set (1). The results in the bottom half of the table summarize predictions by the present methods on 14 Illumina WGS samples (dataset (3)). In a few cases, the present methods offered more complete characterization of the genotype than the validation panels. Although run on PGRNseq samples, Cypiripi is not designed to support such samples and its suboptimal performance is included mainly for reference purposes.

TABLE 6
Summary of CYP2D6 genotypes inferred by the
present methods in comparison to Cypipiri
and Astrolabe on samples from data set (1)

| | Present | | | | |
|---|---|---|---|---|---|
| | Methods | Astrolabe | Cypiripi | Validation | Total |
| PGRNseq v.2 | | | | | |
| Normal | 69 | 68 | 48 | 62 | 69 |
| Deletions | 9 | 0 | 7 | 8 | 9 |
| Duplications | 6 | 0 | 4 | 5 | 6 |
| Fusions | 12 | 0 | 0 | 12 | 12 |
| Total | 96 | 68 | 59 | 87 | 96 |
| Illumina WGS | | | | | |
| Normal | 9 | 3 | 3 | 7 | 9 |
| Duplications | 2 | 0 | 1 | 0 | 2 |
| Fusions | 14 | 0 | 0 | 4 | 14 |
| Total | 25 | 3 | 4 | 11* | 25 |

The summary of the present method's performance on the whole set of 10 ADME genes is provided in Table 7. Table 7 depicts the performance of the present methods on i) 137 PGRNseq v.1 samples from GeT-RM studies (dataset (2)), and on ii) 17 PGRNseq v.2 samples for which had genotype validations were available. More than 99% of the samples were accurately genotyped by the present methods. Furthermore, the present methods found a few instances of novel major star-alleles as well.

TABLE 7
Summary of genotypes for 10 ADME genes inferred by the
present methods on GeT-RM samples (137 PGRNseq v.1 samples)
and Coriell samples (17 PGRNseq v.2 samples).

| | | | Mis- | | |
|---|---|---|---|---|---|
| | Matches | Improvements | matches | Unknown | Total |
| PGRNseq v.1 | | | | | |
| CYP2A6 | 110 | 18 | 3 | 6 | 137 |
| CYP2C8 | 137 | 0 | 0 | 0 | 137 |
| CYP2C9 | 136 | 1 | 0 | 0 | 137 |
| CYP2C19 | 128 | 9 | 0 | 0 | 137 |
| CYP2D6 | 107 | 27 | 0 | 3 | 137 |
| CYP3A4 | 131 | 6 | 0 | 0 | 137 |
| CYP3A5 | 137 | 0 | 0 | 0 | 137 |
| CYP4F2 | 121 | 16 | 0 | 0 | 137 |
| DPYD | 79 | 56 | 0 | 2 | 137 |
| TPMT | 135 | 2 | 0 | 0 | 137 |
| Total | 1221 | 135 | 3 | 11 | 1370 |
| | 89% | 10% | 0.2% | 0.8% | 100% |
| PGRNseq v.2 | | | | | |
| CYP2A6 | 14 | 2 | 0 | 1 | 17 |
| CYP2C8 | 17 | 0 | 0 | 0 | 17 |
| CYP2C9 | 17 | 0 | 0 | 0 | 17 |
| CYP2C19 | 16 | 1 | 0 | 0 | 17 |
| CYP2D6 | 87 | 9 | 0 | 0 | 96 |
| CYP3A4 | 17 | 0 | 0 | 0 | 17 |
| CYP3A5 | 17 | 0 | 0 | 0 | 17 |
| CYP4F2 | 15 | 2 | 0 | 0 | 17 |
| DPYD | 9 | 8 | 0 | 0 | 17 |
| TPMT | 17 | 0 | 0 | 0 | 17 |
| Total | 226 | 22 | 0 | 1 | 249 |
| | 91% | 8.6% | 0% | 0.4% | 100% |

“Matches” column indicates the concordance between the call made by the present methods and panel prediction, while “Mismatches” indicates the discordance. “Improvements” column indicates the potential improvement of calls made by the present methods over the genotyping panels. Finally, “Unknown” indicates the samples for which further validation was needed to clearly call the correct genotype.

In addition to their genotyping accuracy, the methods described herein has very low computational overhead. Each experimental run required less than 10 s and fewer than 100 MB of memory even for the high-coverage PGRNseq samples. Although Astrolabe offers similar performance, it requires VCF data as an input, but generation of such data usually takes significant amounts of system resources. On the other hand, Cypiripi's running time performance significantly drops as the coverage increases and it can take up to 1 h to complete on high-coverage PGRNseq data sets.

As can be seen from Table 6, the present methods are able to accurately identify all CYP2D6 genotypes, and are able to identify all complex events, especially when compared with Astrolabe, which does not support calling such events, and Cypiripi.

Several discrepancies between the present methods and the genotyping panel's predictions were identified. After further investigation, it was concluded that genotyping panels used for validation purposes made either ambiguous or incorrect calls in these cases. Such examples include inability to detect alleles CYP2D6*35 and CYP2D6*45, which were identified as CYP2D6*2 due to the inability of validation kits to detect the discerning SNPs. Additional cases include the misidentification of the CYP2D6*15 allele, which is highly similar to the pseudogene CYP2D7. In some cases, validation kits were unclear about the exact copy number; such samples were cross-validated with the Illumina data and external sources to confirm predictions.

No Mendelian inconsistencies were observed when using the present methods on PGRNseq v.2 and Illumina WGS data. This is in sharp contrast with previous PGRNseq data analysis, which relied on SNP callers to infer genotypes.

Regarding Illumina WGS data, genotypes predicted by the present methods were in accordance with genotypes validated in the literature. Although no genotype information was available for some members of the CEPH 1463 family, predictions made by the present methods were in full accordance with the Mendelian laws of inheritance, as depicted in FIG. 8, which presents a graphical representation of the various structural configurations and their corresponding vectors v. The typical arrangement of CYP2D6 (gene) and CYP2D7 (pseudogene) is indicated at the top of FIG. 8. The first two cases represent the most common configurations, with whole copies of gene and pseudogene, respectively. The third case depicts the conservation of pseudogene's region (in this case, exon 3) within a gene. The fourth case describes a fusion (hybrid) gene. The fifth case depicts partial deletion of the gene, while the sixth case shows how to handle cases when parts of the gene (exon 2 in this case) are duplicated.

The present methods provided accurate genotype calls for other genes as well, as demonstrated in Table 7. In all samples, the accuracy rate was above 99%. A few mismatched calls were mainly due to that unlike PGRNseq v.2, the PGRNseq v.1 reagent does not target the CYP2D6-associated pseudogenes, making accurate genotype calling in the presence of pseudogene-derived mutations significantly harder.

### Novel Alleles

The present methods detected the presence of novel major star-alleles in GeT-RM samples, namely a novel CYP2D6*10-like allele in NA17012 (*10 with c.77 G>, A), novel DPYD alleles (based on either *4, *5, or *6, with c.85 A>G in NA07357, NA10859, and NA24027) and a novel CYP2C19 allele in NA07439 (based on *27 with c.19153 C>T). Furthermore, novel major star-alleles formed by combining variations from two different alleles were observed: in the case of CYP4F2, variations from *2 and *3 allele formed a novel CYP4F2*4 allele, and for the DPYD gene, a novel allele was formed by variations from both *5 and *9 alleles.

In many samples, it was observed that sub-alleles detected by the genotyping methods presented and described herein are not present in the known databases.

A similar observation was made by Raimundo, S. et al. ((2010) Pharmacogenetics 10(7):577-581) regarding the CYP2D6*2 family of sub-alleles. For example, c.843 T>G, associated with all recently discovered *4 sub-alleles (e.g. *4M, *4N and *4P), is not associated with *4 alleles (e.g. *4A, *4B etc.) discovered earlier. However, multiple samples were identified by the present methods, where the evidence indicates that *4A allele contains this SNP. This indicates either the incomplete characterization of *4A sub-allele, or the presence of novel *4 sub-alleles.

The lack of non-functional SNPs can affect the accurate genotype interpretation of HTS-based tools (as already reported by Twist, G. P., et al., (January 2016) npj Genomic Medicine 1:15007). As demonstrated herein, the genotyping methods described herein are capable of identifying additional sub-alleles that can be added to existing databases.

Specialized HTS platforms, such are PGRNseq, are removing the last obstacles preventing the wider integration of HTS technologies in everyday clinical settings. Coupled with fast and accurate genotyping frameworks, such as the ADME genotyping methods describe herein, these platforms can assist physicians in tailoring the prescription recommendations based on a patient's genetic makeup and lead to improved medical care.

While the disclosed subject matter is amenable to various modifications and alternative forms, specific embodiments have been shown by way of example in the drawings and are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step).

1. A method for genotyping a gene, the method comprising:
receiving high throughput sequencing data for the gene from a target sample, wherein the high throughput sequencing data comprises a plurality of target sample reads;
aligning the target sample reads of the high throughput sequencing data to one or more star-alleles of a reference genome allele database, wherein the reference genome allele database comprises nucleic acid sequences for known star-alleles of the gene;
identifying one or more nucleic acid sequence variants, or a lack of nucleic acid variants, in an allele of the gene relative to the one or more star-alleles of the reference genome allele database;
detecting structural variants or a lack of structural variants in the allele;
identifying one or more gene-disrupting mutations or a lack of gene-disrupting mutations in the allele;
selecting a set of one or more star-alleles from the reference genome allele database that most closely match the identified one or more gene-disrupting mutations or a lack of gene-disrupting mutations in the allele; and
calling, for an allele where a single star-allele was selected, a genotype associated with the selected star-allele.

2. The method according to claim 1, wherein the method further comprises refining a genotype for an allele where two or more star-alleles were selected by assigning each of the selected star-alleles a penalty score based on minimizing a number of missing and additional non-functional variations in the allele in order to match the database as closely as possible, and calling a genotype for the allele as that genotype associated with one or more star-alleles having the lowest penalty score.

3. The method according to claim 1, wherein the gene is an absorption, distribution, metabolism, and excretion (ADME) gene.

4. The method according to claim 1, wherein the method is repeated for one or more additional genes.

5. The method according to claim 1, wherein the high throughput data is targeted hybrid capture with consistent read distribution data or whole genome sequencing (WGS) data.

6. The method according to claim 5, wherein the hybrid capture with consistent read distribution data is PGRNseq data.

7. The method according to claim 1, wherein the high throughput data is received as a FASTQ file, a uSAM file, or a uBAM file.

8. The method according to claim 1, wherein alignment of target sample reads of the high throughput sequencing data is accomplished by BWA-MEM, BWA-backtrack, BWA-SW, LAST, Partek Flow, Bowtie 2, Stampy, SHRiMP2, SNP-o-matic, CLC Workbench, NextGenMap, Mosaik, ERNE-MAP, mrFAST, or mrsFAST-Ultra.

9. The method according to claim 1, wherein alignment of target sample reads of the high throughput sequencing data comprises performing a local indel realignment.

10. The method according to claim 1, wherein nucleic acid sequence variants are identified by FreeBayes, MuTect2, or SAMtools.

11. The method according to claim 1, wherein the high throughput sequencing data coverage is consistent across samples but non-uniform across regions of the gene and sequencing depth is not known in advance.

12. The method according to claim 1, wherein detecting structural variations in the allele comprises:
estimating a gene copy number for one or more regions of the allele;
determining an observed coverage for each of the one or more regions; and
identifying an optimal gene arrangement by determining a minimal difference between the observed coverage for each of the one or more regions and coverage formed by one or more known possible gene arrangements,
wherein a structural rearrangement is detected when the optimal gene arrangement is not a reference gene arrangement.

13. The method according to claim 1, wherein identifying gene-disrupting mutations comprises comparing nucleic acid sequence variants identified in the allele and/or structural variation detected in the allele to the known alleles of the reference genome database.

14. The method according to claim 1, wherein selecting a set of star-alleles that most closely match the identified one or more gene-disrupting mutations or lack of gene-disrupting mutations in the allele comprises:
receiving a nucleic acid sequence for each known gene allele of the reference genome database;
excluding nucleic acid sequences of known gene alleles that are not in agreement with a determined allele structural arrangement;
excluding nucleic acid sequences of known gene alleles of the reference genome database that include neutral mutations; and
selecting one or more known gene alleles of the reference genome database that most closely match the identified one or more gene-disrupting mutations or lack of gene-disrupting mutations in the allele.

15. The method according to claim 14, wherein gene structural arrangement is determined by the method of claim 12.

16. The method according to claim 1, wherein the method is executable using a suitably programmed computer.

17. The method according to claim 16, wherein the method according to claim 1 improves the computational capacity of the suitably programmed computer.

18. A system for predicting a genotype of one or more genes, the system comprising:
a sample generator;
a sequencer;
at least one database having information regarding the one or more genes; and
a sequence analyzer comprising a user interface and a system controller comprising at least one processer configured to perform the method according to claim 1.

19. The system of claim 18, wherein the at least one processor comprises a sequence aligner, a sequence variant identifier, a structural variant identifier, a gene-disrupting mutation identifier, a star-allele identifier, and a genotype caller.

20. The system of claim 18, wherein:
the sequence aligner is configured to align target sample reads of the high throughput sequencing data to a reference genome database;
the sequence variant identifier is configured to identify nucleic acid sequence variants in a gene allele relative to the reference genome database;
the structural variant identifier is configured to detect structural variants or a lack of structural variants in the gene allele;
the gene-disrupting mutation identifier is configured to identify one or more gene-disrupting mutations or a lack of gene-disrupting mutations in the gene allele;
the star-allele identifier is configured to identify one or more star-alleles corresponding to the identified one or more gene-disrupting mutations or lack of gene-disrupting mutations; and
the genotype caller is configured to determine the allele to have the genotype associated with the identified one or more star-alleles.

21. The system according to claim 18, wherein elements of the system are integrated into a standalone system located at a single site.

22. The system according to claim 18, wherein one or more elements of the system are located remotely with respect to each other.

23. A system for predicting a genotype of one or more genes, the system comprising:
at least one database having information regarding the one or more genes; and
a sequence analyzer comprising a user interface and a system control comprising at least one processor configured to perform the method according to claim 1, wherein the at least one processor comprises a sequence aligner, a sequence variant identifier, a structural variant identifier, a gene-disrupting mutation identifier, a star-allele identifier, and a genotype caller.
